(19) United States
(12) Patent Application Publication
(10) Pub. No.: US 2026/0317328 A1
(43) Pub. Date: Oct. 15, 2026

(54) SMALL MOLECULES FOR ADOPTIVE T-CELL THERAPY (ACT) THROUGH ACTIVATION OF THE mTOR SIGNALLING PATHWAY, PROCESS FOR PREPARATION THEREOF

(71) Applicant: COUNCIL OF SCIENTIFIC & INDUSTRIAL RESEARCH, New Delhi (IN)

(72) Inventors: Arindam TALUKDAR, Kolkata (IN); Shilpak CHATTERJEE, Kolkata (IN); Sunny GOON, Kolkata (IN); Dipika SARKAR, Kolkata (IN); Puspendu GHOSH, Kolkata (IN); Uddipta Ghosh DASTIDAR, Kolkata (IN); Trisha GHOSH, Kolkata (IN)

(21) Appl. No.: 19/485,106

(22) PCT Filed: Apr. 24, 2024
(86) PCT No.: PCT/IN2024/050437

§ 371 Date: Nov. 17, 2025

(30) Foreign Application Priority Data
May 18, 2023 (IN) 202311034981

(51) Int. Cl. C07D 473/34; A61K 31/52; A61K 40/11; A61P 35/00

(52) U.S. Cl. CPC C07D 473/34; A61K 31/52; A61K 40/11; A61P 35/00

(57) ABSTRACT

The present invention relates to the preparation of 8-phenyl-purine-6-amino compounds with general structure I in free form or in acceptable salt form to identify small molecule activator of mTOR, which can be used to augment the effector function (IFNγ production) of T cells and hence heightened their antitumor response. More particularly, the invention relates to small molecules where R1, R2 and R3 are as defined in the description, capable of IFNγ production via mTOR activation pathway to elicit durable antitumor repose in the host. The invention describes strategies to generate T cells with high effector function, yet in a relatively short period of time so that a robust and durable anti-tumor response can be achieved in the host.

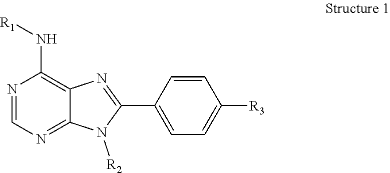

### FIELD OF THE INVENTION

The present invention relates to the preparation of 8-phenyl-purine-6-amino compounds with general structure I in free form or in acceptable salt form to identify small molecule activator of mTOR, which can be used to augment the effector function (IFNγ production) of T cells and hence heightened their antitumor response. More particularly, the invention relates to small molecules where R₁, R₂ and R₃ are as defined in the description, capable of IFNγ production via mTOR activation pathway to elicit durable antitumor repose in the host.

### BACKGROUND OF THE INVENTION

Immunotherapy has revolutionized the therapeutic management of cancers. In recent years, Food and Drug Administration (FDA) has approved a number of immune checkpoint inhibitors (ICIs) for clinical management of more than 85 malignancies. However, a large fraction of patients do not benefit from ICI due, in part, to the scarcity of tumor-specific effector T cells. This limitation could be overcome with ACT, which involves infusion of antigen-specific T cells in the cancer bearing host (Waldman et al., Nat Rev Immunol 20, 651-668 (2020). Different ACT techniques are being developed, including tumor-infiltrating lymphocyte (TIL) therapy, T cell receptor-engineered T (TCR-T) cell therapy, and chimeric antigen receptor T (CAR-T) cell therapy. The ACT initially developed was based on the isolation of tumor-specific TILs for ex vivo expansion and reinfusion into the patient. While effective in certain cancer types such as melanoma, this approach was only feasible for resectable tumors from which enough T cells could be isolated and amplified. TCR-T and CAR-T cell therapies consist in genetically engineered T cells, modified to express a receptor directed against a tumor antigen. Although, ACT have shown remarkable responses in some patients with malignancy, in majority of cases the response is less rewarding. One of the major obstacles in obtaining objective response with ACT is suboptimum effector function of transferred T cells in the tumor-bearing host. This is primarily attributed to the prolong expansion of tumor reactive T cells ex vivo before being infused back into the patients for ACT. It has been shown that rapid expansion protocol which is usually employed to expand the T cells for ACT severely affect the effector function of the T cells and makes the T cells highly susceptible to activation induced cell death (AICD). Therefore, a strategy to boost up effector function of T cells is of utmost importance for successful application of ACT in clinics.

Reference may be made to article by Powell et al., Immunity 33, 301-311 (2010), wherein mammalian target of rapamycin (mTOR), an evolutionarily conserved ser/thr kinase, is considered as an important metabolic rheostat regulating T cell differentiation and effector function. The mTOR forms two structurally and functionally distinct multicompetent complexes called mTORC1 and mTORC2.

Reference may be made to article by Zonku et al., Nature Rev. Mol. Cell Biol. 12, 21-35 (2011), wherein mTORC1 is primarily regulates cell growth and metabolism. mTORC2 mainly controls cell proliferation and survival. In T cells, activation of mTOR has been found to be indispensable in generating effector response.

Reference may be made to article by Thomson et al., Nature Rev. Immunol. 9, 324-337 (2009), wherein mTORC1 promotes the differentiation of Th1 and Th17 response, whereas mTORC2 promotes Th2 differentiation. Moreover, initial transient inhibition of mTOR activity in CD4⁺ T cells is found to be necessary for population expansion of regulatory T cells (Treg) in vitro. Reference may be made to article by Araki et al. Nature 460, 108-112, 2009, wherein mTORC1 utilizes multiple mechanisms to influence effector versus memory CD8⁺ T cell differentiation and function.

Reference may be made to article by Sowell et al. J Immunol, 2067-71, 2014, wherein critical role for mTOR in modulating tissue-specific, effector versus memory fate decisions in CD8⁺ T cells disclosed.

Reference may be made to article by Zou et al., J Cell Biol. 105-22, 2015, wherein activation of mTORC1 also sustains the activity of various downstream signalling molecules involves in regulating T cell metabolism and effector function differentiation. For example, activity of HIF1α is stabilized in mTORC1 dependent manner during Th17 differentiation. Likewise, the sustained upregulation of c-Myc is also dependent on mTORC1 after T cell activation. Both HIF1α and c-Myc have shown to promote glycolytic and glutaminolytic pathways, not only further fuels the activation of mTOR signalling in a feed-forward regulatory loop, but also facilitate the differentiation of T cells with long lasting effector function.

Thus, keeping in view the drawbacks of the hitherto reported prior arts, there was a need to solve the problem of providing “Sustained activation of mTOR in anti-tumor CD8+ T cells during initial activation, that will promote the generation of long lasting and highly effector T cells and elicit durable anti-tumor repose in the host”.

### OBJECTIVES OF THE INVENTION

The main objective of present invention is to identify small molecule activator of mTOR, which can be used to augment the effector function (IFNγ production) of T cells and hence heightened their anti-tumor response.

Another objective of the present invention is to preparation of 8-phenyl-purine-6-amino with general structure I in free form or in acceptable salt form to identify small molecule activator of mTOR, which can be used to augment the effector function (IFNγ production) of T cells and hence heightened their antitumor response Another objective of the present invention is to evaluate the potential of small molecule activator of mTOR in improving the metabolic fitness of T cells.

Yet another objective of the present invention is to evaluate the potential of mTOR activator in modulating the expression of immune checkpoint inhibitors (PD1, Tim3, Lag3, CD38) on T cells.

Yet another objective of the present invention is to evaluate the efficacy of mTOR activator in generating T cells with improved anti-tumor response in preclinical models.

Yet another objective of the present invention is to evaluate the efficacy of mTOR activator-induced anti-tumor T cells in generating long-lasting anti-tumor response in tumor bearing mice.

### SUMMARY OF THE INVENTION

In an embodiment of the present invention relates to the 8-phenyl-purine-6-amino compounds represented by following structure I or a pharmaceutically acceptable salt thereof:

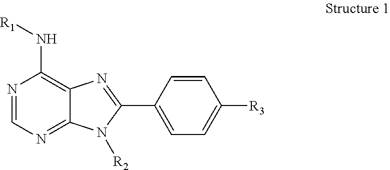

Wherein
R₁ is independently selected from groups referred to as follows:

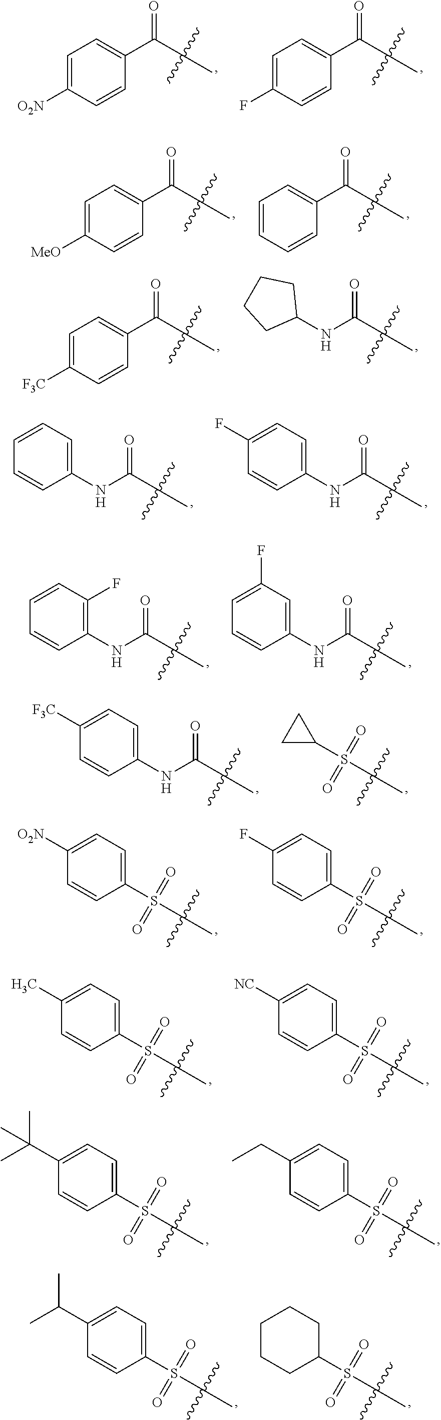

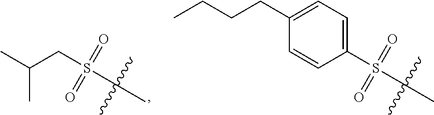

R₂ is independently selected from groups referred to as follows:

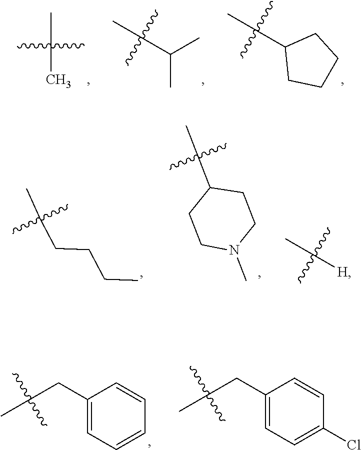

R₃ is independently selected from groups referred to as follows:
—OMe, —Br, —Cl, —CF₃, —OCF₃, —CH₃, —F, —CN
   The 8-Phenyl-Purine-6-Amino Compounds of Structure I Selected from the Group Consisting of:
N-(8-(4-methoxyphenyl)-9-methyl-9H-purin-6-yl)-4-nitrobenzamide (5)
4-fluoro-N-(8-(4-methoxyphenyl)-9-methyl-9H-purin-6-yl)benzamide (6)
4-methoxy-N-(8-(4-methoxyphenyl)-9-methyl-9H-purin-6-yl)benzamide (7)
N-(8-(4-methoxyphenyl)-9-methyl-9H-purin-6-yl)benzamide (8)
N-(8-(4-methoxyphenyl)-9-methyl-9H-purin-6-yl)-4-(trifluoromethyl)benzamide (9)
1-cyclopentyl-3-(8-(4-methoxyphenyl)-9-methyl-9H-purin-6-yl)urea (10)
1-(8-(4-methoxyphenyl)-9-methyl-9H-purin-6-yl)-3-phenylurea (11)
1-(4-fluorophenyl)-3-(8-(4-methoxyphenyl)-9-methyl-9H-purin-6-yl)urea (12)
1-(2-fluorophenyl)-3-(8-(4-methoxyphenyl)-9-methyl-9H-purin-6-yl)urea (13)
1-(3-fluorophenyl)-3-(8-(4-methoxyphenyl)-9-methyl-9H-purin-6-yl)urea (14)
1-(8-(4-methoxyphenyl)-9-methyl-9H-purin-6-yl)-3-(4-(trifluoromethyl)phenyl)urea (15)
N-(8-(4-methoxyphenyl)-9-methyl-9H-purin-6-yl)cyclopropanesulfonamide (16)
N-(8-(4-methoxyphenyl)-9-methyl-9H-purin-6-yl)-4-nitrobenzenesulfonamide (17)
4-fluoro-N-(8-(4-methoxyphenyl)-9-methyl-9H-purin-6-yl)benzenesulfonamide (18)
N-(8-(4-methoxyphenyl)-9-methyl-9H-purin-6-yl)-4-methylbenzenesulfonamide (19)
4-cyano-N-(8-(4-methoxyphenyl)-9-methyl-9H-purin-6-yl)benzenesulfonamide (20)
4-(tert-butyl)-N-(8-(4-methoxyphenyl)-9-methyl-9H-purin-6-yl)benzenesulfonamide (21)
4-ethyl-N-(8-(4-methoxyphenyl)-9-methyl-9H-purin-6-yl)benzenesulfonamide (22)
4-isopropyl-N-(8-(4-methoxyphenyl)-9-methyl-9H-purin-6-yl)benzenesulfonamide (23)
N-(8-(4-methoxyphenyl)-9-methyl-9H-purin-6-yl)cyclohexanesulfonamide (24)
N-(8-(4-methoxyphenyl)-9-methyl-9H-purin-6-yl)-2-methylpropane-1-sulfonamide (25)
4-butyl-N-(8-(4-methoxyphenyl)-9-methyl-9H-purin-6-yl)benzenesulfonamide (26)
N-(9-isopropyl-8-(4-methoxyphenyl)-9H-purin-6-yl)-4-methylbenzenesulfonamide (30)
N-(9-cyclopentyl-8-(4-methoxyphenyl)-9H-purin-6-yl)-4-methylbenzenesulfonamide (32)
N-(9-butyl-8-(4-methoxyphenyl)-9H-purin-6-yl)-4-methylbenzenesulfonamide (34)
N-(8-(4-methoxyphenyl)-9-(1-methylpiperidin-4-yl)-9H-purin-6-yl)-4-methylbenzenesulfonamide (36)
N-(8-(4-methoxyphenyl)-9H-purin-6-yl)-4-methylbenzenesulfonamide (37)
N-(8-(4-fluorophenyl)-9-methyl-9H-purin-6-yl)-4-methylbenzenesulfonamide (39)
N-(8-(4-chlorophenyl)-9-methyl-9H-purin-6-yl)-4-methylbenzenesulfonamide (41)
N-(8-(4-cyanophenyl)-9-methyl-9H-purin-6-yl)-4-methylbenzenesulfonamide (43)
N-(9-benzyl-8-(4-methoxyphenyl)-9H-purin-6-yl)-4-methylbenzenesulfonamide (44)
N-(9-(4-chlorobenzyl)-8-(4-methoxyphenyl)-9H-purin-6-yl)-4-methylbenzenesulfonamide (45)

Another embodiment of the present invention provides a process for the preparation of 8-phenyl-purine-6-amino compounds having structure I, wherein the steps comprising:
(i) Adenine (Compound 1) was taken in (1:1) Dioxane& water mixture and NaOH was added. Then TBAF was added in the reaction mixture. Next Mel was added dropwise at 0° C. temperature and the reaction mixture was allowed to stir at room temperature for overnight to 2 as off-white solid.
(ii) Compound 2 was dissolved in a (9:1) mixture of Dioxane & 0.5(M) NaOAc solution. Then liq. Bromine was added dropwise in reaction mixture and stirred at room temperature for overnight to 3 as pale yellow amorphous solid.
(iii) Compound 3, required boronic acids (4-methoxy phenyl boronic acid, 4-fluoro phenyl boronic acid, 4-chloro phenyl boronic acid, 4-cyano phenyl boronic acid), K₂CO₃ were taken in a pressure tube and dissolved in (9:1) mixture of Dioxane& water. Solution was purged with Argon gas for 30 minutes. Then catalyst Pd(PPh₃)₄ was added and the reaction mixture was stirred at 100° C.-110° C. for overnight to get compounds 4, 38, 40, 42 as pale yellow amorphous solid.
(iv) Treating the compound 4 obtained in step (iii) with LiHMDS as a base followed by reaction with an acid chloride selected from the group consisting 4-nitrobenzoyl chloride, 4-fluorobenzoyl chloride, 4-methoxybenzoyl chloride, benzoyl chloride, 4-(trifluoromethyl)benzoyl chloride in dry THF at 0° C. temperature for 30 minutes to obtain the compound with amide linkage having structure I selected from the group consisting of 5, 6, 7, 8, 9.
(v) Treating the compound 4 obtained in step (iii) with LiHMDS as a base followed by reaction with an isocyanate selected from the group consisting isocyanatocyclopentane, Phenyl isocyanate, 4-Fluorophenyl isocyanate, 2-Fluorophenyl isocyanate, 3-Fluorophenyl isocyanate, 4-(Trifluoromethyl)phenylisocyanate in dry THF at 0° C. temperature for 30 minutes to obtain the compound with urea linkage having structure I selected from the group consisting of 10, 11, 12, 13, 14, 15.
(vi) Treating the compound 4 obtained in step (iii) with NaH as a base followed by reaction with an sulfonyl chloride selected from the group consisting cyclopropanesulfonyl chloride, 4-nitrobenzene-1-sulfonyl chloride, 4-fluorobenzene-1-sulfonyl chloride, 4-methylbenzene-1-sulfonyl chloride, 4-cyanobenzene-1-sulfonyl chloride, 4-(tert-butyl)benzene-1-sulfonyl chloride, 4-ethylbenzene-1-sulfonyl chloride, 4-isopropylbenzene-1-sulfonyl chloride, cyclohexanesulfonyl chloride, 2-methylpropane-1-sulfonyl chloride, 4-butylbenzene-1-sulfonyl chloride in dry DMF at 0° C. temperature for 30 minutes to obtain the compound with sulphonamide linkage having structure I selected from the group consisting of 16, 17, 18, 19, 20, 21, 22, 23, 24, 25, 26.
(vii) Treating the compound 38, 40, 42 obtained in step (iii) with NaH as a base followed by reaction with 4-methylbenzene-1-sulfonyl chloride in dry DMF at 0° C. temperature for 30 minutes to obtain the compound with sulphonamide linkage having structure I selected from the group consisting of 39, 41, 43.
(viii) Adenine (Compound 1) was taken in dry DMF and K₂CO₃ was added. Next required aliphatic or aromatic halide such as isopropyl iodide, cyclopentyl bromide, benzyl bromide, 4-chlorobenzyl bromide was added into the reaction mixture and stirred for 12 hrs to get compounds 1A, 1C, 1E respectively.
(ix) Treating the compound 1A, 1C, 1E obtained in step (viii) with NBS in dry CHCl3 for 12 hrs at room temperature provided compounds 1B, 1D, 1F respectively.
(x) Compounds 1B, 1D, 1F (as required) and 4-methoxy phenyl boronic acid, K₂CO₃ were taken in a pressure tube and dissolved in (9:1) mixture of Dioxane & water. Solution was purged with Argon gas for 30 minutes. Then catalyst Pd(PPh₃)₄ was added and the reaction mixture was stirred at 100° C.-110° C. for overnight to get compounds 29, 31, 1G as pale yellow amorphous solid.
(xi) Treating the compound 29, 31, 1G obtained in step (x) with NaH as a base followed by reaction with 4-methylbenzene-1-sulfonyl chloride in dry DMF at 0° C. temperature for 30 minutes to obtain the compound with sulphonamide linkage having structure I selected from the group consisting of 30, 32, 44, 45.

In another embodiment of the present invention, wherein 8-phenyl-purine-6-amino compounds with structure I are capable to increase the effector function (IFNγ production) of T cells and hence heightened their anti-tumor response.

In another embodiment of the present invention, 8-phenyl-purine-6-amino compounds of structure I used in modulating the expression of immune checkpoint inhibitors (PD1, Tim3, Lag3, CD38) on T cells.

Yet another embodiment of the present invention, wherein 8-phenyl-purine-6-amino compounds of structure I provides a method to establish a relation between mTOR activation and optimal T-cell effector function and process evaluate the efficacy of mTOR activator in generating T cells with improved anti-tumor response in preclinical models.

Yet another embodiment of the present invention, wherein 8-phenyl-purine-6-amino compounds with structure I in the present invention can induce anti-tumor T cells and can generate long-lasting anti-tumor response in tumor bearing mice.

Yet another embodiment of the present invention, wherein 8-phenyl-purine-6-amino compounds with structure I used in cancer immunotherapy especially in adoptive T cell therapy (ACT) to enhance T cell effector functionality and also in cases where T cell activation is useful.

### BRIEF DESCRIPTION OF DRAWING

The invention has other advantages and features which will be more readily apparent from the following detailed description of the invention and the appended claims, when taken in conjunction with the accompanying drawings, in which:

FIG. 1 (A-B): Initial small molecule screening:

To select the small molecule capable of inducing IFN-γ production in CD8⁺ T cells, mouse and CD3⁺ sorted naïve T cells were activated with each small molecule for 72 hrs followed by an intracellular cytokine staining. Amongst the small molecules, Comp-19 was found to be most promising in terms of improving effector cytokine production from CD8⁺ T cells. Our experimental results proved that, IFN-γ and TNF-α production was highest from CD8⁺ T cells when activated in presence of 10 μM Comp-19 compared to vehicle control/other small molecules treated group (FIG. 1A-B).

FIG. 2 (A-E): Metabolic and effector function of CD8⁺ cells improved by Comp-19:

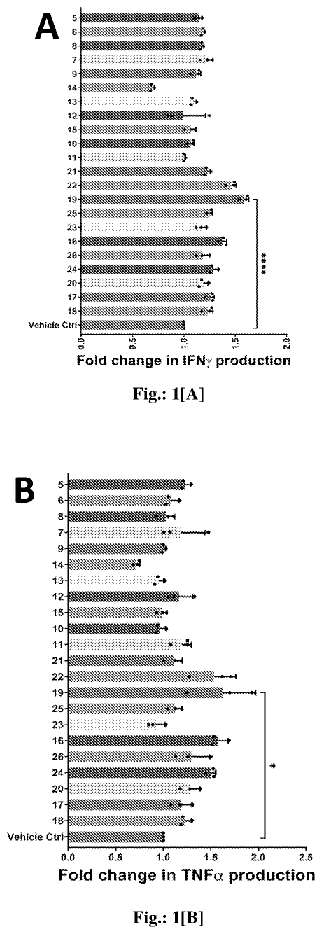

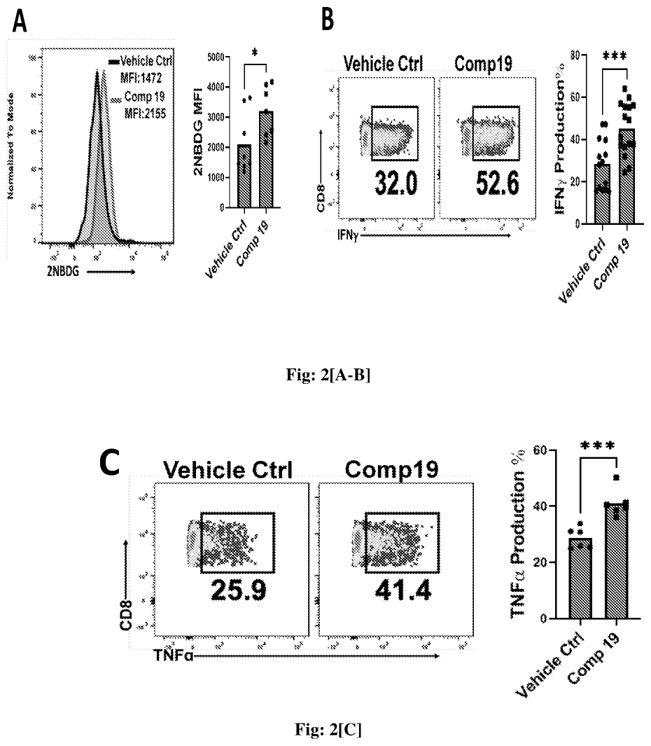

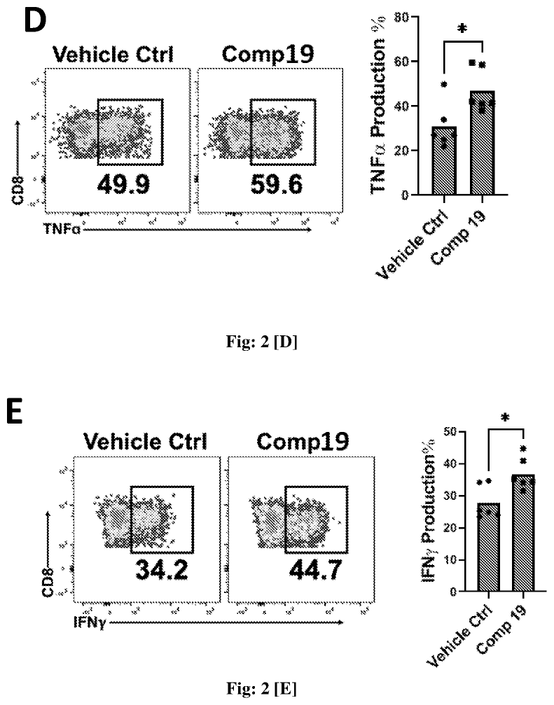

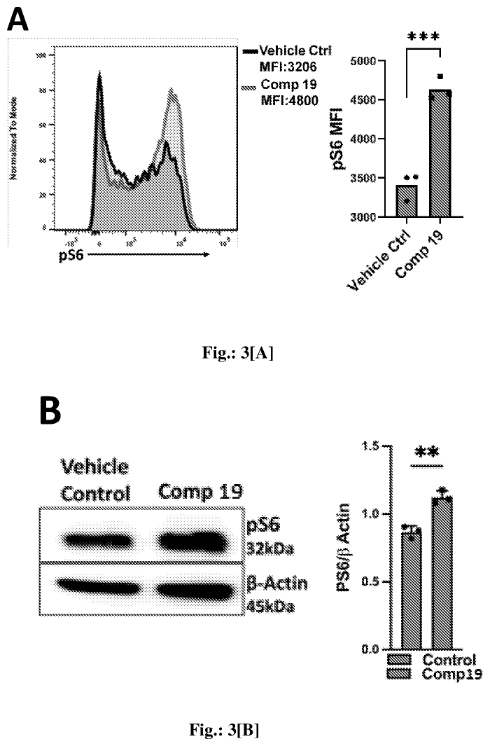

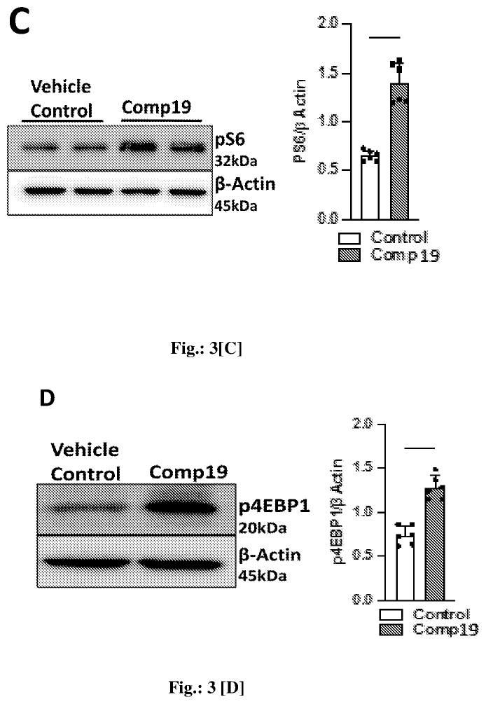

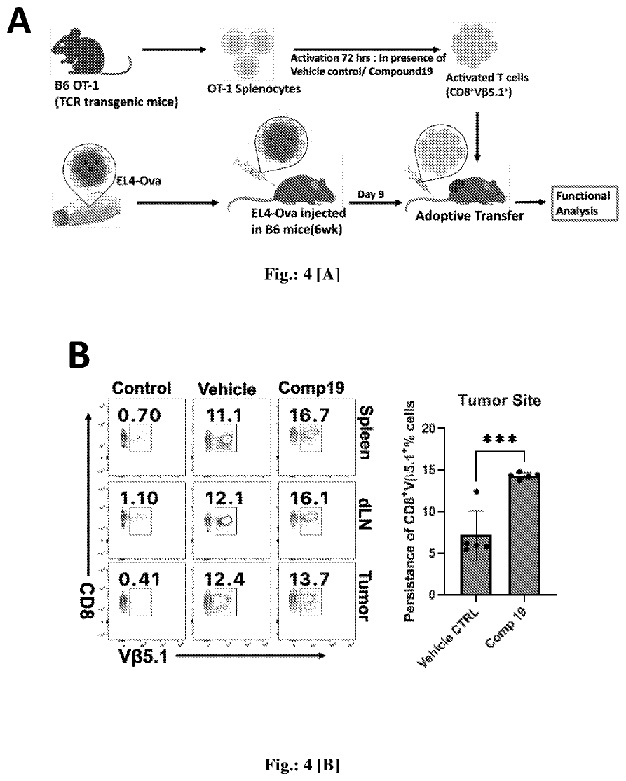

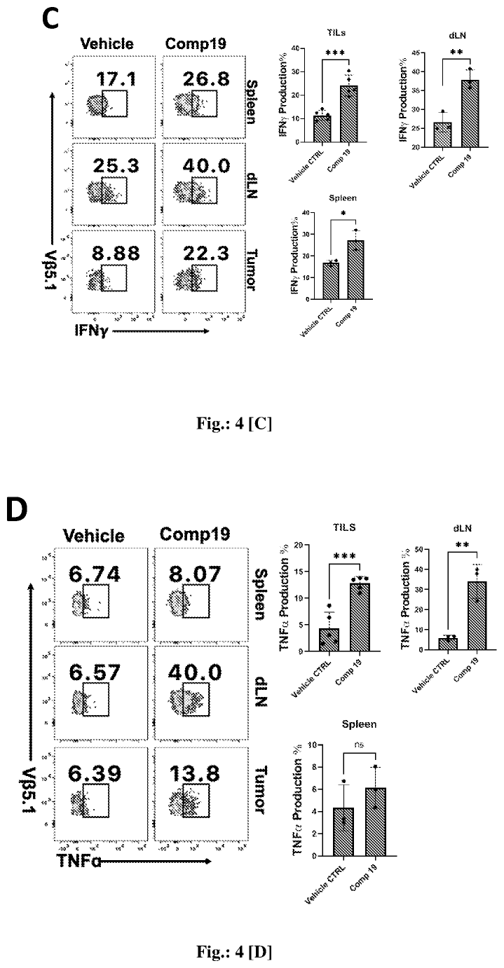

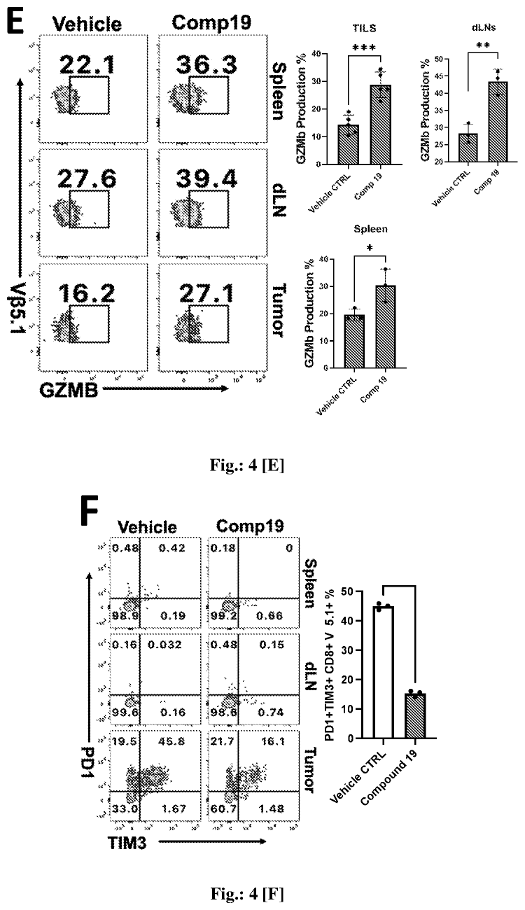

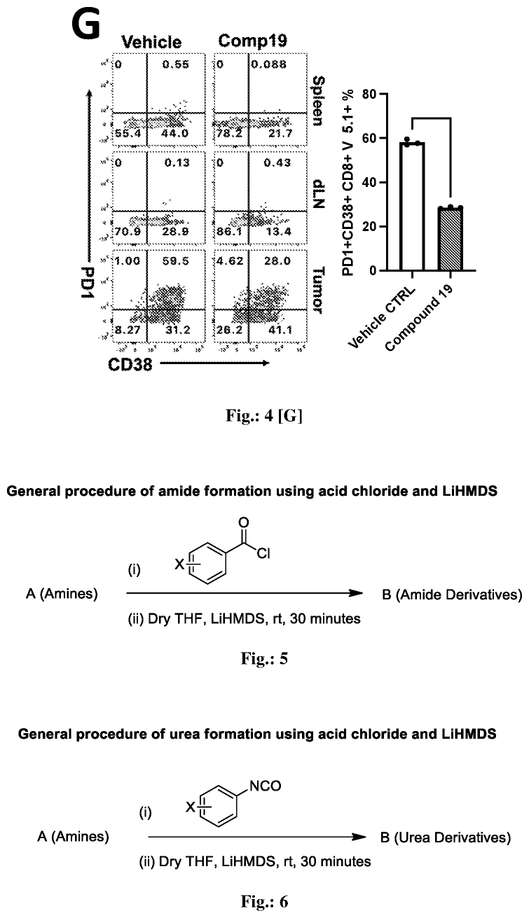

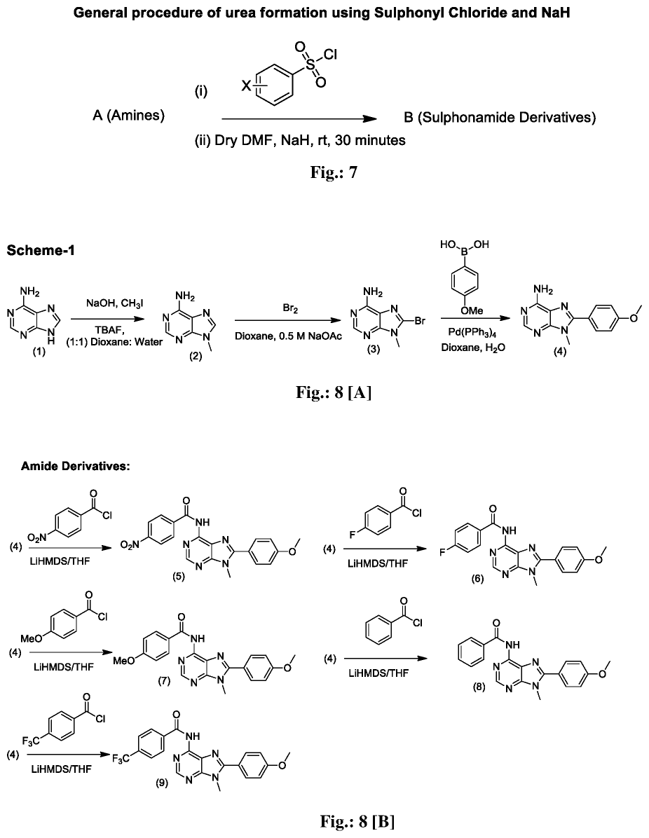

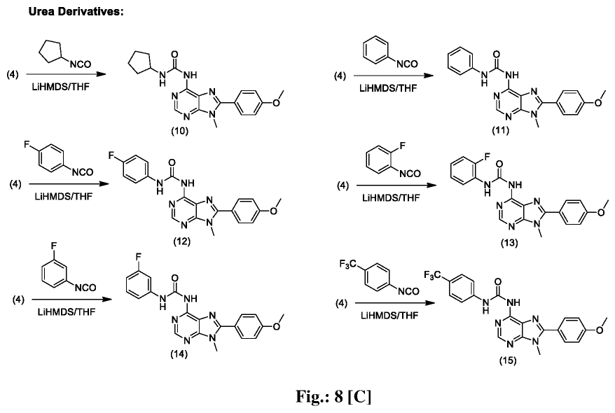

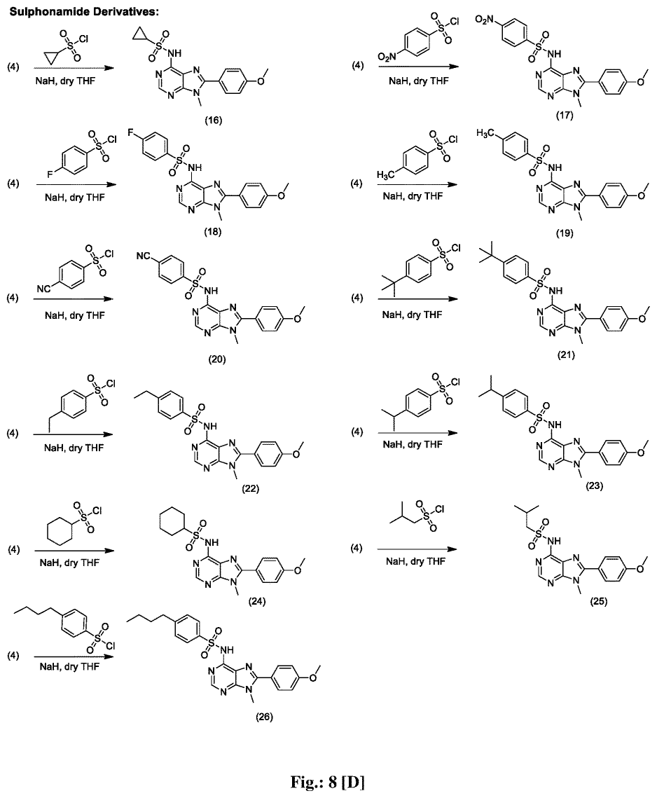

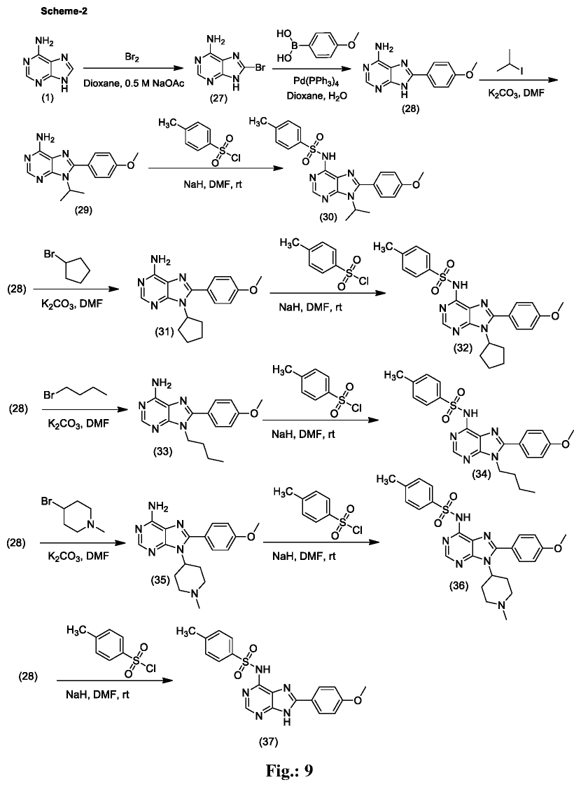

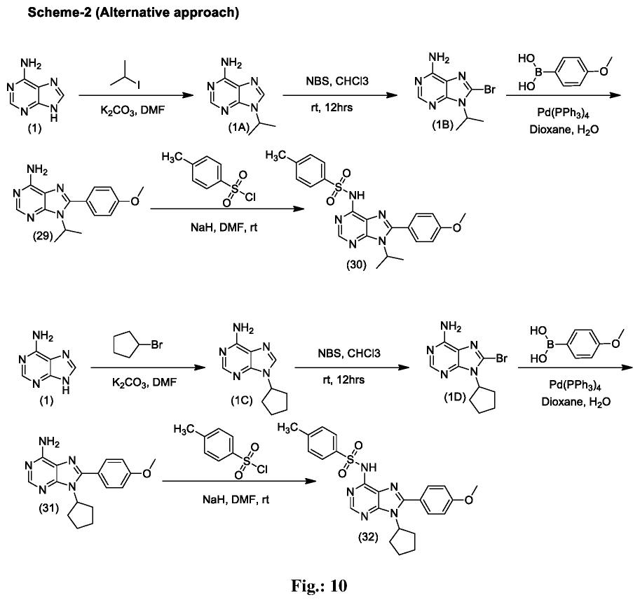

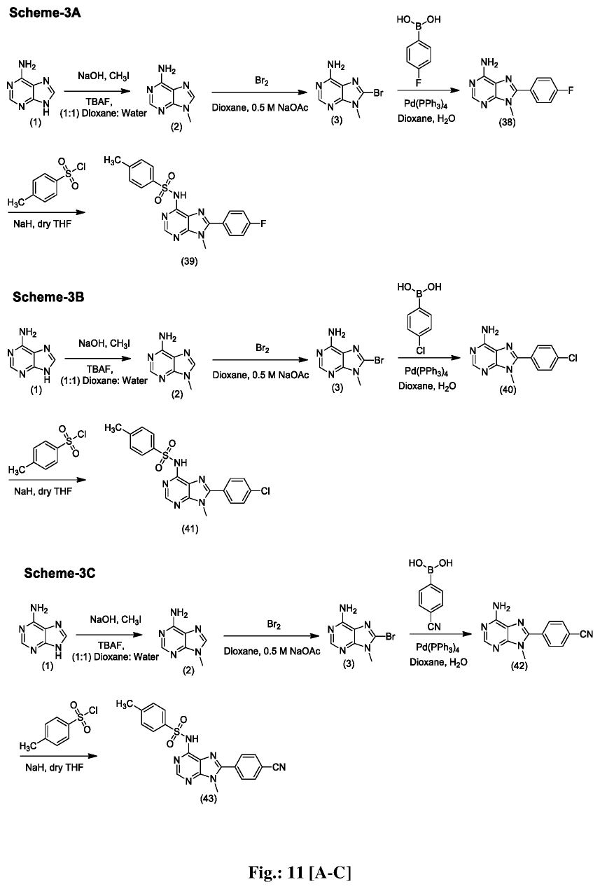

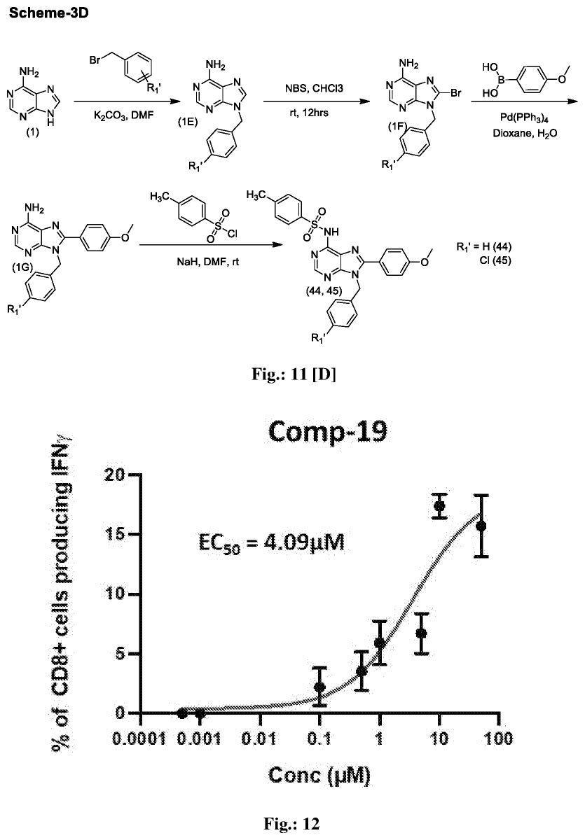

Distinct T cell states require specific metabolic programs compatible to their functional demands. The transition between various T cell states is accompanied by active rewiring of cellular metabolism and epigenetic landscape. Oxidative phosphorylation is the main biochemical pathway that drives metabolism of naïve T cell. Upon activation, naïve T cells rapidly reprogram their metabolic networks to comply with the demands of robust clonal expansion and epigenetic remodeling. So, TCR activation and co-stimulation initiate a set of signaling cascades that collectively license a T cell to exit quiescence. Stimulation of the TCR promotes signaling through the ERK/MAPK pathways and calcium flux, activation of the PI3K-AKT-mTOR axis which subsequently resulting in T cell activation. This conversion to an activated effector T cell is marked by enhanced glucose uptake, engagement of aerobic glycolysis, increased oxidative phosphorylation. So, we assessed glucose uptake level by activated CD8⁺ T cells using fluorescent labeled deoxy-glucose analogue, 2NBDG. It was seen that activating naïve T cells in presence of Comp-19 not only promoted glucose uptake (FIG. 2A) but also increased their cytolytic function marked by significant increase in IFN-γ and TNF-α production level from CD8⁺ T cell (FIG. 2B-E).

FIG. 3 (A-D): Comp-19 promotes mTORC1 activity in CD8+ T cells:

Several transcription factors and signaling pathways coordinately support and regulate the metabolic shifting of naïve T cell after activation. Downstream of co-stimulation and PI3K-AKT signaling, mammalian target of Rapamycin (mTOR) kinase pathway helps in integrating multiple signals, plays a significant role for cell cycle entry and coordinates early anabolic changes that occur upon T cell activation mTOR complex 1 (mTORC1) promote aerobic glycolysis and support effector T cell proliferation, growth, and cytokine production. Translation repressor protein 4E-BP1 inhibits cap-dependent translation by binding to the translation initiation factor eIF4E. Phosphorylation of 4E-BP1 by mTORC1 disrupts this interaction and results in activation of cap-dependent translation. On the contrary, phosphorylation of S6 ribosomal protein correlates with an increase in translation of mRNA transcripts that contain an oligopyrimidine tract in their 5′ untranslated regions. Activation of mTORC1 increases protein translation via direct phosphorylation of 4E-BP1 and p70S6 kinase. Since Comp-19 was proposed as mTORC1 activator, next we sought to check expression level ofpS6 and p4EBP1 level inside CD8⁺ T cells activated in presence/absence of Comp-19. Flow cytometry analysis and western blot data indicated a marked increase in pS6 level inside. CD8⁺ T cells treated with Comp-19 (FIG. 3A-C). Similarly, the other downstream substrate of mTORC1, 4EBP1 also exhibited a significant high expression upon Comp-19 treatment (FIG. 3D), Hence our experimental results proved Comp-19 as a potent mTOR-Cl activator improving metabolic fitness and effector function of CD8⁺ T cell.

FIG. 4 (A-G): Comp-19 promotes antitumor efficacy of CD8⁺ T cells:

Given the functional fitness of Comp-19 treated CD8⁺ T cells in vitro, we, next, sought to determine their antitumor potential upon adoptive transfer into mice bearingEL4-OVA tumors. It was found that adoptive transfer of OT1 T cells (0.5×106 cells/mouse) activated in the presence of 10 μMml⁻¹ Comp-19 could efficiently control tumor growth, and markedly enhanced the survival of tumor-bearing mice. Comparing the frequency of tumor epitope-reactive circulating T cells (CD8⁺Vβ5.1⁺OT1 T cells) in tumor-bearing host revealed a higher number of CD8⁺Vβ5.1⁺ T cells in mice who received Comp-19 treated cells as compared with mice who received DMSO treated cells. Furthermore, our data suggested that the enhanced tumor control observed with CD8+Vβ5.1+OT1 T cells was correlated with their reduced expression of inhibitory receptors i.e., PD1, TIM3, CD38 (FIG. 4F,4G) improved persistence (FIG. 4B) and ability to produce IFN-γ, TNF-α (FIG. 4C-E) at the tumor site.

FIG. 5: General procedure of amide formation using acid chloride and LiHMDS.

FIG. 6: General procedure of urea formation using acid chloride and LiHMDS.

FIG. 7: General procedure of urea formation using sulphonyl chloride and NaH.

FIG. 8(A-D): Scheme 1.

FIG. 9: Scheme 2.

FIG. 10: Scheme 2 (Alternative approach)

FIG. 11: Scheme 3A-D.

FIG. 12: EC₅₀ data of Comp-19:

Human naïve CD8⁺ T cells were sorted from PBMC and activated in presence of Comp 19 in the different doses (500 pM, 1 nM, 100 nM, 500 nM, 1 μM, 5 μM, 10 μM, 50 μM). After 72 hrs, activated CD8+ T cells were restimulated and stained for IFNγ production. Percent of CD8+ T cell producing IFNγ in the previously mentioned doses of Comp 19 was normalized to a vehicle control (DMSO) treated group. The optimum dosage of Comp 19 for inducing effector cytokine (IFNγ) production from CD8⁺ T cells is 10 μM. Calculated EC50 is 4.09 μM. (N=3 for each dose of Comp 19).

### DETAILED DESCRIPTION OF THE INVENTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein. In line with the above objectives, The present invention relates to a the 8-phenyl-purine-6-amino compounds of structure I or a salt thereof:

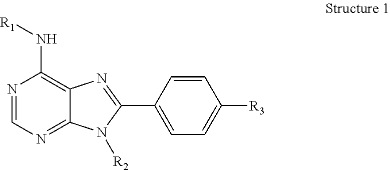

Wherein
R₁ is independently selected from groups referred to as follows:

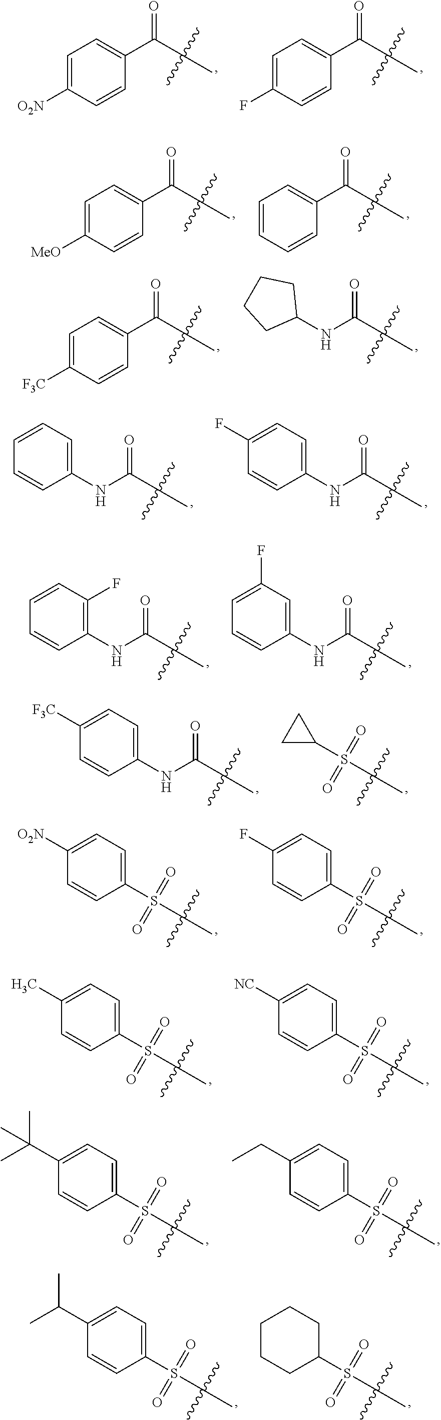

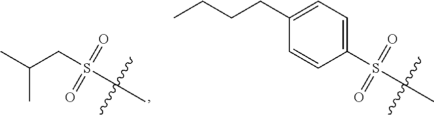

R₂ is independently selected from groups referred to as follows:

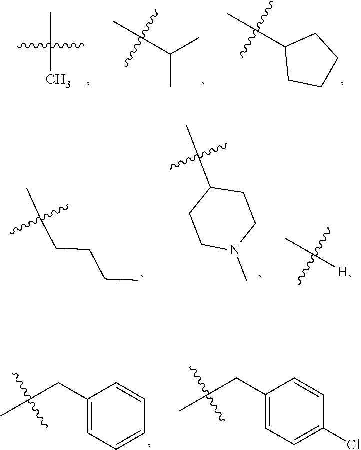

R₃ is independently selected from groups referred to as follows:
—OMe, —Br, —Cl, —CF₃, —OCF₃, —CH₃, —F, —CN

### The Compound of Present Invention has the Structure of Structure I as Depicted in the Table Below:

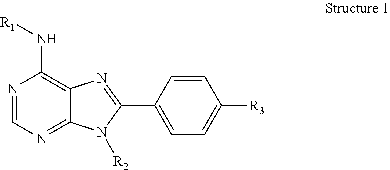

TABLE 1
Structure of the 8-phenyl-purine-6-amino compounds

| | | | | | |
|---|---|---|---|---|---|
| Comp. | | | | | |
| No | R₁ | R₂ | R₃ | Structure | IUPAC Name |

| | | | | | |
|---|---|---|---|---|---|
| 5 | 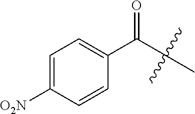 [SMILES: *C(=O)c1ccc([N+](=O)[O-])cc1] | CH₃ | OMe | 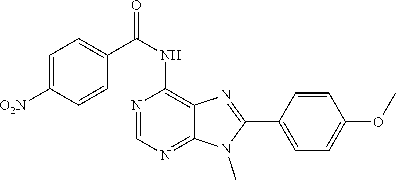 | N-(8-(4- methoxyphenyl)-9- methyl-9H-purin-6- yl)-4-nitrobenzamide |
| 6 | 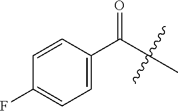 [SMILES: *C(=O)c1ccc(F)cc1] | CH₃ | OMe | 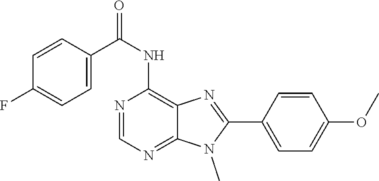 | 4-fluoro-N-(8-(4- methoxyphenyl)-9- methyl-9H-purin-6- yl)benzamide |
| 7 | 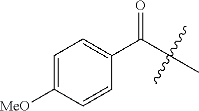 [SMILES: *C(=O)c1ccc(OC)cc1] | CH₃ | OMe | 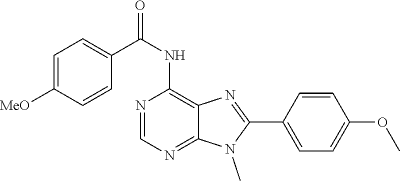 | 4-methoxy-N-(8-(4- methoxyphenyl)-9- methyl-9H-purin-6- yl)benzamide |
| 8 | 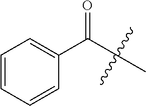 [SMILES: *C(=O)c1ccccc1] | CH₃ | OMe | 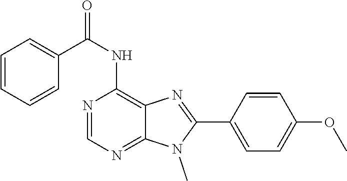 | N-(8-(4- methoxyphenyl)-9- methyl-9H-purin-6- yl)benzamide |
| 9 | 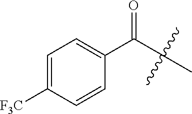 [SMILES: *C(=O)c1ccc(C(F)(F)F)cc1] | CH₃ | OMe | 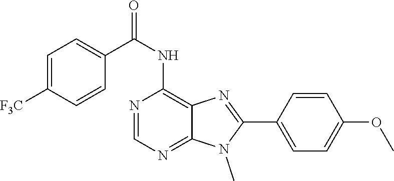 | N-(8-(4- methoxyphenyl)-9- methyl-9H-purin-6- yl)-4- (trifluoromethyl) benzamide |
| 10 | 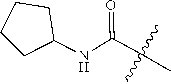 [SMILES: *C(=O)NC1CCCC1] | CH₃ | OMe | 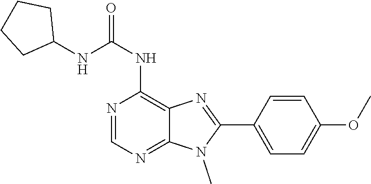 | 1-cyclopentyl-3-(8- (4-methoxyphenyl)-9- methyl-9H-purin-6- yl)urea |
| 11 | 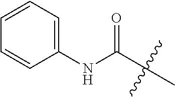 [SMILES: *C(=O)Nc1ccccc1] | CH₃ | OMe | 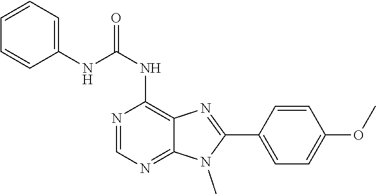 | 1-(8-(4- methoxyphenyl)-9- methyl-9H-purin-6- yl)-3-phenylurea |
| 12 | 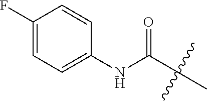 [SMILES: *C(=O)Nc1ccc(F)cc1] | CH₃ | OMe | 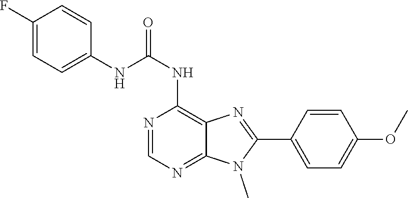 | 1-(4-fluorophenyl)-3- (8-(4- methoxyphenyl)-9- methyl-9H-purin-6- yl)urea |
| 13 | 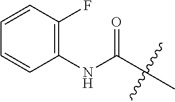 [SMILES: *C(=O)Nc1ccccc1F] | CH₃ | OMe | 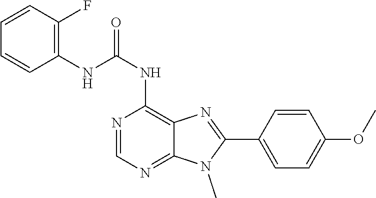 | 1-(2-fluorophenyl)-3- (8-(4- methoxyphenyl)-9- methyl-9H-purin-6- yl)urea |
| 14 | 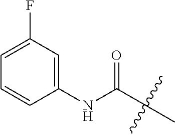 [SMILES: *C(=O)Nc1cccc(F)c1] | CH₃ | OMe | 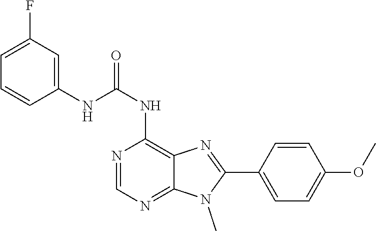 | 1-(3-fluorophenyl)-3- (8-(4- methoxyphenyl)-9- methyl-9H-purin-6- yl)urea |
| 15 | 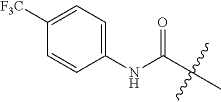 [SMILES: *C(=O)Nc1ccc(C(F)(F)F)cc1] | CH₃ | OMe | 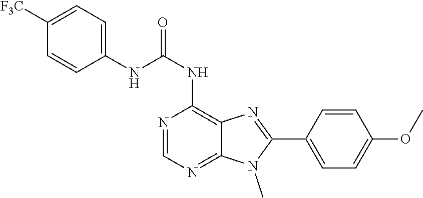 | 1-(8-(4- methoxyphenyl)-9- methyl-9H-purin-6- yl)-3-(4- (trifluoromethyl) phenyl)urea |
| 16 | 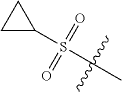 [SMILES: *S(=O)(=O)C1CC1] | CH₃ | OMe | 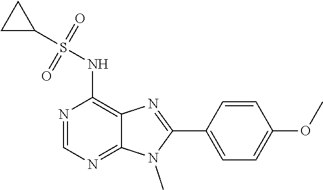 | N-(8-(4- methoxyphenyl)-9- methyl-9H-purin-6- yl)cyclopropane- sulfonamide |
| 17 | 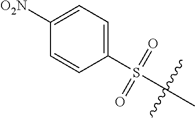 [SMILES: *S(=O)(=O)c1ccc([N+](=O)[O-])cc1] | CH₃ | OMe | 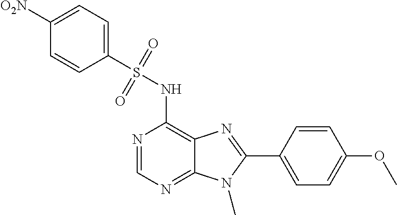 | N-(8-(4- methoxyphenyl)-9- methyl-9H-purin-6- yl)-4- nitrobenzenesulfonamide |
| 18 | 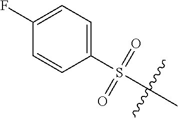 [SMILES: *S(=O)(=O)c1ccc(F)cc1] | CH₃ | OMe | 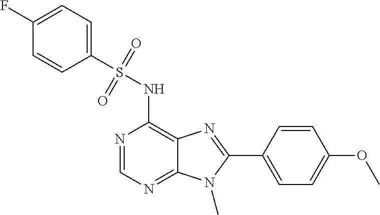 | 4-fluoro-N-(8-(4- methoxyphenyl)-9- methyl-9H-purin-6- yl)benzenesulfonamide |
| 19 | 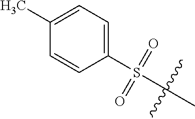 [SMILES: *S(=O)(=O)c1ccc(C)cc1] | CH₃ | OMe | 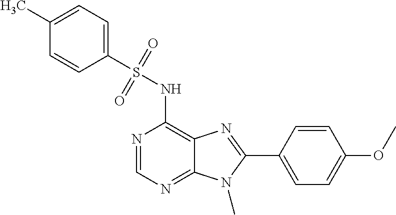 | N-(8-(4- methoxyphenyl)-9- methyl-9H-purin-6- yl)-4-methyl- benzenesulfonamide |
| 20 | 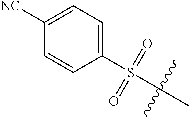 [SMILES: *S(=O)(=O)c1ccc(C#N)cc1] | CH₃ | OMe | 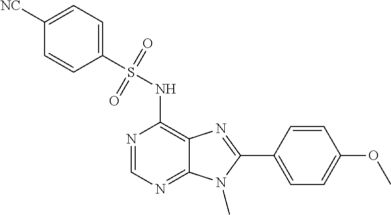 | 4-cyano-N-(8-(4- methoxyphenyl)-9- methyl-9H-purin-6- yl)benzenesulfonamide |
| 21 | 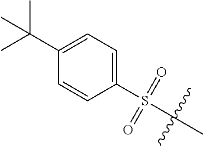 [SMILES: *S(=O)(=O)c1ccc(C(C)(C)C)cc1] | CH₃ | OMe | 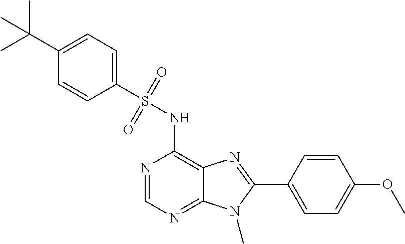 | 4-(tert-butyl)-N-(8-(4- methoxyphenyl)-9- methyl-9H-purin-6- yl)benzenesulfonamide |
| 22 | 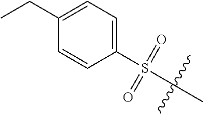 [SMILES: *S(=O)(=O)c1ccc(CC)cc1] | CH₃ | OMe | 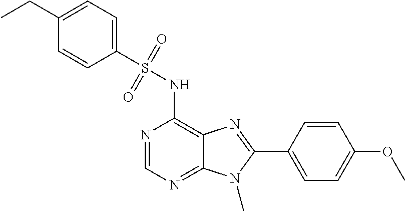 | 4-ethyl-N-(8-(4- methoxyphenyl)-9- methyl-9H-purin-6- yl)benzenesulfonamide |
| 23 | 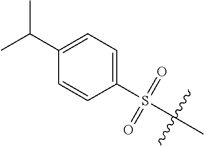 [SMILES: *S(=O)(=O)c1ccc(C(C)C)cc1] | CH₃ | OMe | 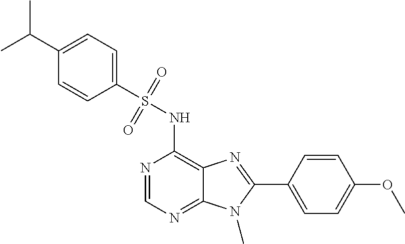 | 4-isopropyl- N-(8-(4- methoxyphenyl)-9- methyl-9H-purin-6- yl)benzenesulfonamide |
| 24 | 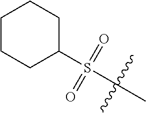 [SMILES: *S(=O)(=O)C1CCCCC1] | CH₃ | OMe | 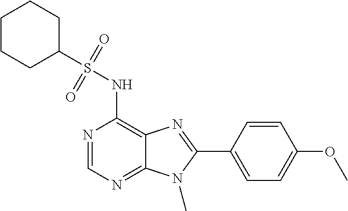 | N-(8-(4- methoxyphenyl)-9- methyl-9H-purin-6- yl)cyclohexane- sulfonamide |
| 25 | 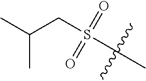 [SMILES: *S(=O)(=O)CC(C)C] | CH₃ | OMe | 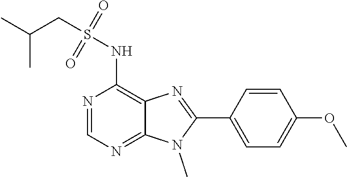 | N-(8-(4- methoxyphenyl)-9- methyl-9H-purin-6- yl)-2-methylpropane- 1-sulfonamide |
| 26 | 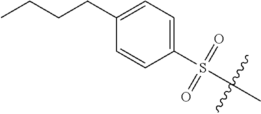 [SMILES: *S(=O)(=O)c1ccc(CCCC)cc1] | CH₃ | OMe | 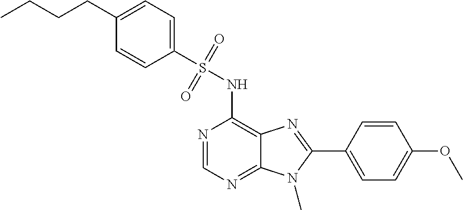 | 4-butyl-N-(8-(4- methoxyphenyl)-9- methyl-9H-purin-6- yl)benzenesulfonamide |
| 30 | 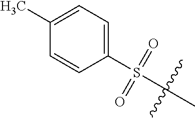 [SMILES: *S(=O)(=O)c1ccc(C)cc1] | 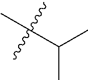 [SMILES: *C(C)C] | OMe | 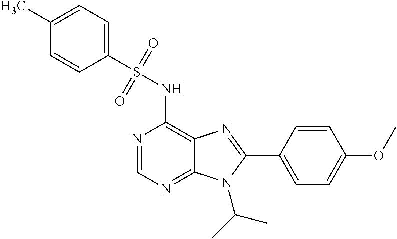 | N-(9-isopropyl-8-(4- methoxyphenyl)-9H- purin-6-yl)-4- methylbenzene- sulfonamide |
| 32 | 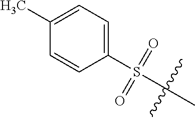 [SMILES: *S(=O)(=O)c1ccc(C)cc1] | 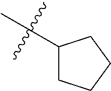 [SMILES: *C1CCCC1] | OMe | 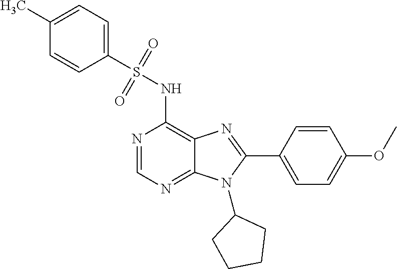 | N-(9-cyclopentyl-8- (4-methoxyphenyl)- 9H-purin-6-yl)-4- methylbenzene- sulfonamide |
| 34 | 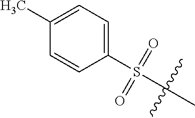 [SMILES: *S(=O)(=O)c1ccc(C)cc1] | 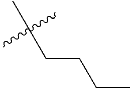 [SMILES: *CCCC] | OMe | 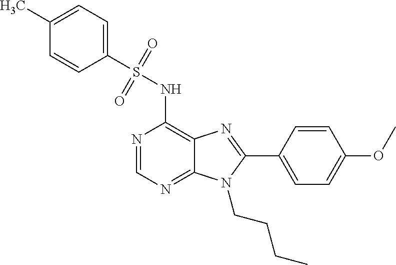 | N-(9-butyl-8-(4- methoxyphenyl)-9H- purin-6-yl)-4- methylbenzene- sulfonamide |
| 36 | 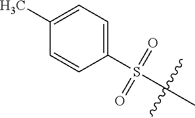 [SMILES: *S(=O)(=O)c1ccc(C)cc1] | 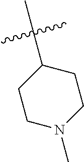 [SMILES: *C1CCN(C)CC1] | OMe | 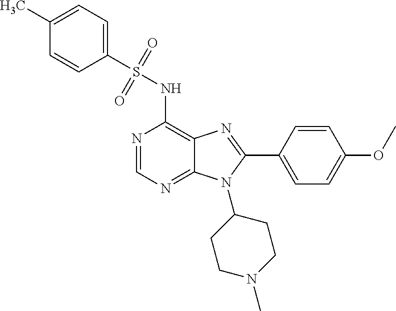 | N-(8-(4- methoxyphenyl)-9-(1- methylpiperidin-4- yl)-9H-purin-6-yl)-4- methylbenzene- sulfonamide |
| 37 | 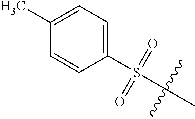 [SMILES: *S(=O)(=O)c1ccc(C)cc1] | H | OMe | 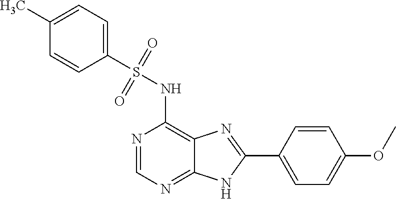 | N-(8-(4- methoxyphenyl)-9H- purin-6-yl)-4- methylbenzene- sulfonamide |
| 39 | 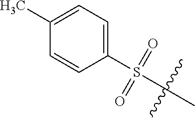 [SMILES: *S(=O)(=O)c1ccc(C)cc1] | CH₃ | Br | 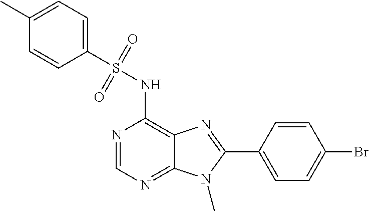 | N-(8-(4- bromophenyl)-9- methyl-9H-purin-6- yl)-4-methyl- benzenesulfonamide |
| 40 | 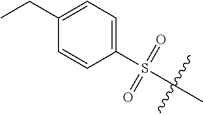 [SMILES: *S(=O)(=O)c1ccc(CC)cc1] | CH₃ | Br | 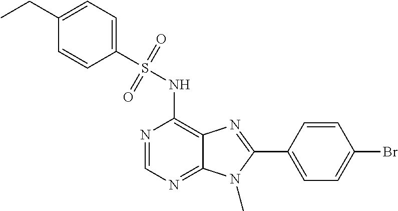 | N-(8-(4- bromophenyl)-9- methyl-9H-purin-6- yl)-4-ethyl- benzenesulfonamide |
| 42 | 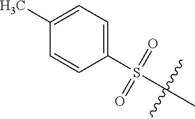 [SMILES: *S(=O)(=O)c1ccc(C)cc1] | CH₃ | Cl | 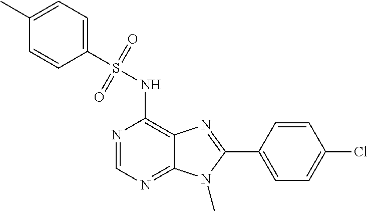 | N-(8-(4- chlorophenyl)-9- methyl-9H-purin-6- yl)-4-methyl- benzenesulfonamide |
| 43 | 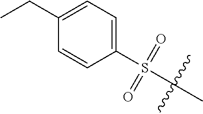 [SMILES: *S(=O)(=O)c1ccc(CC)cc1] | CH₃ | Cl | 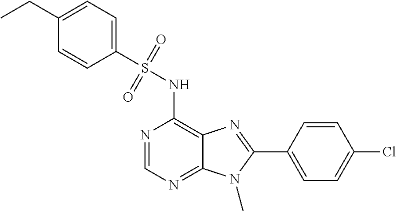 | N-(8-(4- chlorophenyl)-9- methyl-9H-purin-6- yl)-4-ethyl- benzenesulfonamide |
| 45 | 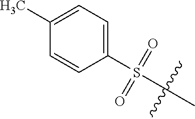 [SMILES: *S(=O)(=O)c1ccc(C)cc1] | CH₃ | CF₃ | 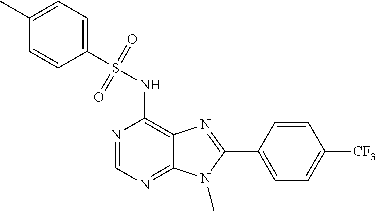 | 4-methyl-N-(9- methyl-8-(4- (trifluoromethyl) phenyl)-9H-purin-6- yl)benzene- sulfonamide |
| 46 | 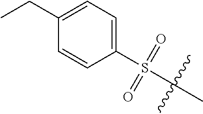 [SMILES: *S(=O)(=O)c1ccc(CC)cc1] | CH₃ | CF₃ | 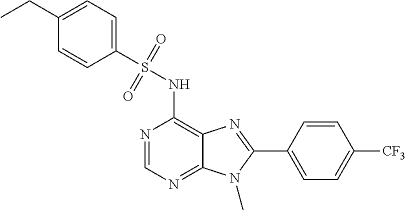 | 4-ethyl-N-(9-methyl- 8-(4- (trifluoromethyl) phenyl)-9H-purin-6- yl)benzene- sulfonamide |
| 48 | 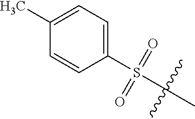 [SMILES: *S(=O)(=O)c1ccc(C)cc1] | CH₃ | CH₃ | 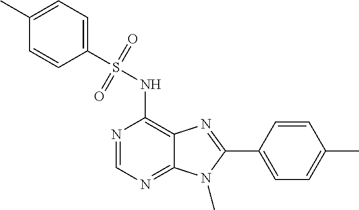 | 4-methyl-N-(9- methyl-8-(p-tolyl)- 9H-purin-6- yl)benzene- sulfonamide |
| 49 | 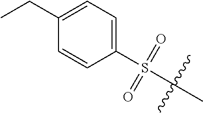 [SMILES: *S(=O)(=O)c1ccc(CC)cc1] | CH₃ | CH₃ | 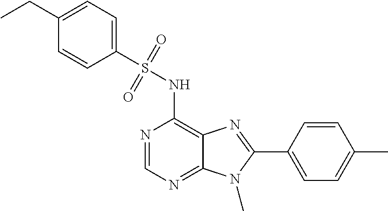 | 4-ethyl-N-(9-methyl- 8-(p-tolyl)-9H-purin- 6-yl) benzenesulfonamide |
| 51 | 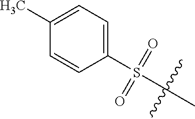 [SMILES: *S(=O)(=O)c1ccc(C)cc1] | CH₃ | OCF₃ | 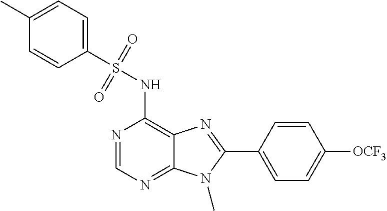 [SMILES: Cc1ccc(S(=O)(=O)Nc2ncnc3c2nc(-c2ccc(OC(F)(F)F)cc2)n3C)cc1] | 4-methyl-N-(9- methyl-8-(4- (trifluoromethoxy) phenyl)-9H-purin-6- yl)benzene- sulfonamide |
| 52 | 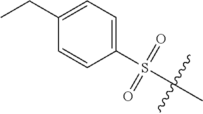 [SMILES: *S(=O)(=O)c1ccc(CC)cc1] | CH₃ | OCF₃ | 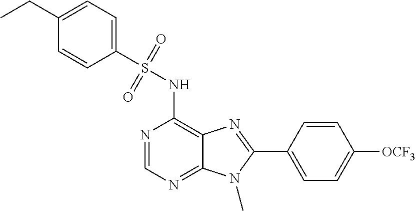 [SMILES: CCc1ccc(S(=O)(=O)Nc2ncnc3c2nc(-c2ccc(OC(F)(F)F)cc2)n3C)cc1] | 4-ethyl-N-(9-methyl- 8-(4- (trifluoromethoxy) phenyl)-9H-purin-6- yl)benzene- sulfonamide |
| 53 | 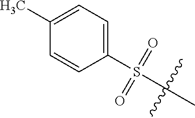 [SMILES: *S(=O)(=O)c1ccc(C)cc1] | CH₃ | F | 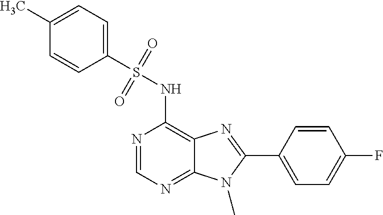 | N-(8-(4- fluorophenyl)-9- methyl-9H-purin-6- yl)-4-methyl- benzenesulfonamide |
| 54 | 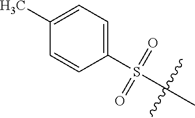 [SMILES: *S(=O)(=O)c1ccc(C)cc1] | CH₃ | CN | 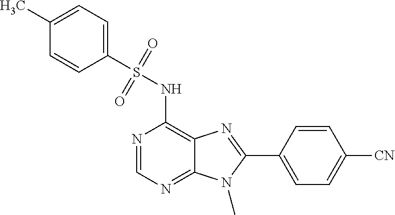 | N-(8-(4- cyanophenyl)-9- methyl-9H-purin-6- yl)-4-methyl- benzenesulfonamide |
| 55 | 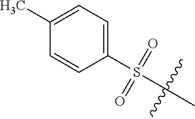 [SMILES: *S(=O)(=O)c1ccc(C)cc1] | 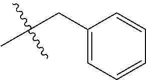 [SMILES: *Cc1ccccc1] | OMe | 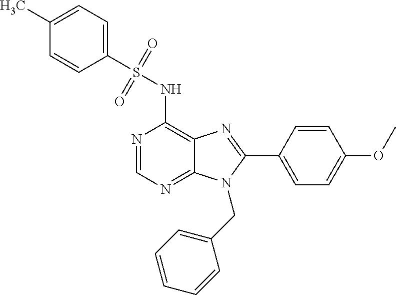 | N-(9-benzyl-8-(4- methoxyphenyl)-9H- purin-6-yl)-4- methylbenzene- sulfonamide |
| 56 | 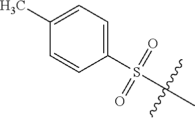 [SMILES: *S(=O)(=O)c1ccc(C)cc1] | 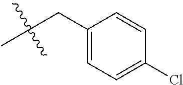 [SMILES: *Cc1ccc(Cl)cc1] | OMe | 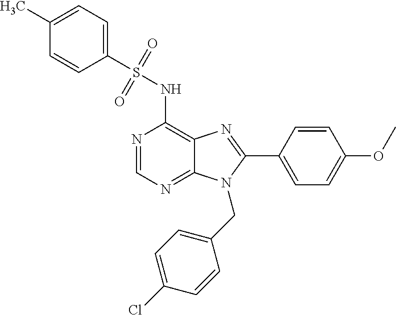 | N-(9-(4- chlorobenzyl)-8-(4- methoxyphenyl)-9H- purin-6-yl)-4- methylbenzene- sulfonamide |

### General Process of Preparation:

(i) Adenine (compound 1) was taken in (1:1) Dioxane& water mixture and NaOH was added. Then TBAF was added in the reaction mixture. Next Mel was added dropwise at 0° C. temperature and the reaction mixture were allowed to stir at room temperature for overnight to 2 as off-white solid.
(ii) Compound 2 was dissolved in a (9:1) mixture of Dioxane&0.5(M) NaOAc solution. Then liq.bromine was added dropwise in reaction mixture and stirred at room temperature for overnight to 3 as pale yellow amorphous solid.
iii) Compound 3, required boronic acids (4-methoxy phenyl boronic acid, 4-fluoro phenyl boronic acid, 4-chloro phenyl boronic acid, 4-cyano phenyl boronic acid), K2CO3 were taken in a pressure tube and dissolved in (9:1) mixture of Dioxane& water. Solution was purged with Argon gas for 30 minutes. Then catalyst Pd(PPh3)4 was added and the reaction mixture was stirred at 100° C.-110° C. for overnight to get compounds 4, 38, 40, 42 as pale yellow amorphous solid.
(iv) Treating the compound 4 obtained in step (iii) with LiHMDS as a base followed by reaction with an acid chloride selected from the group consisting 4-nitrobenzoyl chloride, 4-fluorobenzoyl chloride, 4-methoxybenzoyl chloride, benzoyl chloride, 4-(trifluoromethyl)benzoyl chloride in dry THF at 0° C. temperature for 30 minutes to obtain the compound with amide linkage having structure I selected from the group consisting of 5, 6, 7, 8, 9.
(v) Treating the compound 4 obtained in step (iii) with LiHMDS as a base followed by reaction with an isocyanate selected from the group consisting isocyanatocyclopentane, Phenyl isocyanate, 4-Fluorophenyl isocyanate, 2-Fluorophenyl isocyanate, 3-Fluorophenyl isocyanate, 4-(Trifluoromethyl)phenylisocyanate in dry THF at 0° C. temperature for 30 minutes to obtain the compound with urea linkage having structure I selected from the group consisting of 10, 11, 12, 13, 14, 15.
(vi) Treating the compound 4 obtained in step (iii) with NaH as a base followed by reaction with an sulfonyl chloride selected from the group consisting cyclopropanesulfonyl chloride, 4-nitrobenzene-1-sulfonyl chloride, 4-fluorobenzene-1-sulfonyl chloride, 4-methylbenzene-1-sulfonyl chloride, 4-cyanobenzene-1-sulfonyl chloride, 4-(tert-butyl)benzene-1-sulfonyl chloride, 4-ethylbenzene-1-sulfonyl chloride, 4-isopropylbenzene-1-sulfonyl chloride, cyclohexanesulfonyl chloride, 2-methylpropane-1-sulfonyl chloride, 4-butylbenzene-1-sulfonyl chloridein dry DMF at 0° C. temperature for 30 minutes to obtain the compound with urea linkage having structure I selected from the group consisting of 16, 17, 18, 19, 20, 21, 22, 23, 24, 25, 26.
(vii) Treating the compound 38, 40, 42 obtained in step (iii) with NaH as a base followed by reaction with 4-methylbenzene-1-sulfonyl chloride in dry DMF at 0° C. temperature for 30 minutes to obtain the compound with sulphonamide linkage having structure I selected from the group consisting of 39, 41, 43.
(viii) Adenine (Compound 1) was taken in dry DMF and K2CO3 was added. Next required aliphatic or aromatic halide such as isopropyl iodide, cyclopentyl bromide, benzyl bromide, 4-chlorobenzyl bromide was added into the reaction mixture and stirred for 12 hrs to get compounds 1A, 1C, 1E respectively.
(ix) Treating the compound 1A, 1C, 1E obtained in step (viii) with NBS in dry CHCl3 for 12 hrs at room temperature provided compounds 1B, 1D, 1F respectively.
(x) Compounds 1B, 1D, 1F (as required) and 4-methoxy phenyl boronic acid, K₂CO₃ were taken in a pressure tube and dissolved in (9:1) mixture of Dioxane & water. Solution was purged with Argon gas for 30 minutes. Then catalyst Pd(PPh₃)₄ was added and the reaction mixture was stirred at 100° C.-110° C. for overnight to get compounds 29, 31, 1G as pale yellow amorphous solid.
(xi) Treating the compound 29, 31, 1G obtained in step (x) with NaH as a base followed by reaction with 4-methylbenzene-1-sulfonyl chloride in dry DMF at 0° C. temperature for 30 minutes to obtain the compound with sulphonamide linkage having structure I selected from the group consisting of 30, 32, 44, 45.

Table 2 provide the structure of reactants and products obtained with reaction using acid chloride and LiHMDS

TABLE 2

| Serial No | Amine (A) | 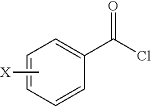 | B (Amide compounds) |
|---|---|---|---|
| 1 | 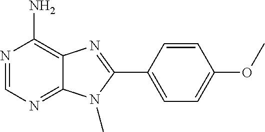 [SMILES: COc1ccc(-c2nc3c(N)ncnc3n2C)cc1] (4) | 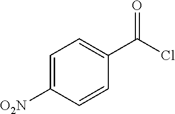 [SMILES: O=C(Cl)c1ccc([N+](=O)[O-])cc1] | 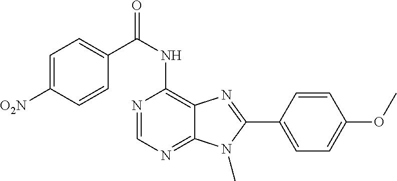 [SMILES: COc1ccc(-c2nc3c(NC(=O)c4ccc([N+](=O)[O-])cc4)ncnc3n2C)cc1] |
| 2 | 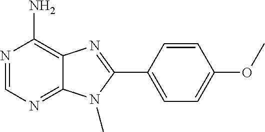 [SMILES: COc1ccc(-c2nc3c(N)ncnc3n2C)cc1] (4) | 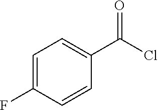 | 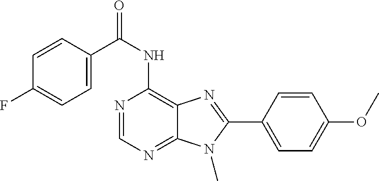 [SMILES: COc1ccc(-c2nc3c(NC(=O)c4ccc(F)cc4)ncnc3n2C)cc1] |
| 3 | 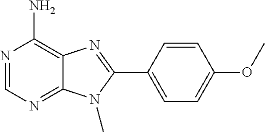 [SMILES: COc1ccc(-c2nc3c(N)ncnc3n2C)cc1] (4) | 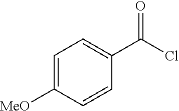 [SMILES: COc1ccc(C(=O)Cl)cc1] | 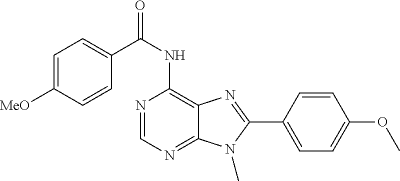 [SMILES: COc1ccc(C(=O)Nc2ncnc3c2nc(-c2ccc(OC)cc2)n3C)cc1] |
| 4 | 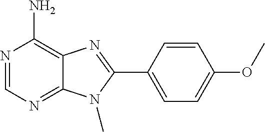 [SMILES: COc1ccc(-c2nc3c(N)ncnc3n2C)cc1] (4) | 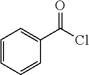 | 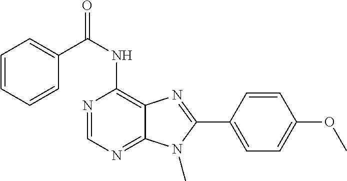 [SMILES: COc1ccc(-c2nc3c(NC(=O)c4ccccc4)ncnc3n2C)cc1] |
| 5 | 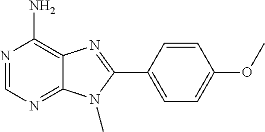 [SMILES: COc1ccc(-c2nc3c(N)ncnc3n2C)cc1] (4) | 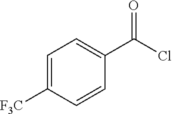 [SMILES: O=C(Cl)c1ccc(C(F)(F)F)cc1] | 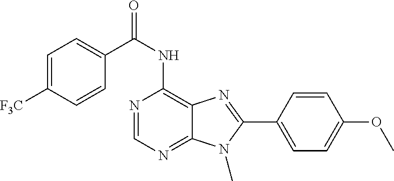 [SMILES: COc1ccc(-c2nc3c(NC(=O)c4ccc(C(F)(F)F)cc4)ncnc3n2C)cc1] |

Table 3 provide the structure of reactants and products obtained with reaction using isocyanates and LiHMDS

TABLE 3

| Serial No | Amine (A) | 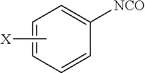 | B (Amide compounds) |
|---|---|---|---|
| 1 | 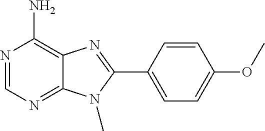 [SMILES: COc1ccc(-c2nc3c(N)ncnc3n2C)cc1] (4) | 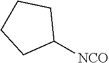 | 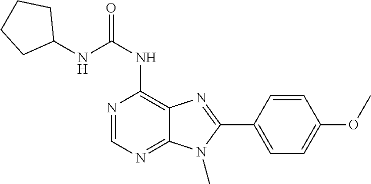 [SMILES: COc1ccc(-c2nc3c(NC(=O)NC4CCCC4)ncnc3n2C)cc1] |
| 2 | 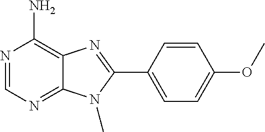 [SMILES: COc1ccc(-c2nc3c(N)ncnc3n2C)cc1] (4) | 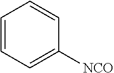 | 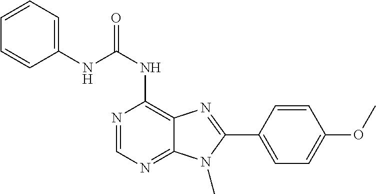 [SMILES: COc1ccc(-c2nc3c(NC(=O)Nc4ccccc4)ncnc3n2C)cc1] |
| 3 | 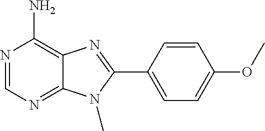 [SMILES: COc1ccc(-c2nc3c(N)ncnc3n2C)cc1] (4) | 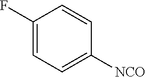 | 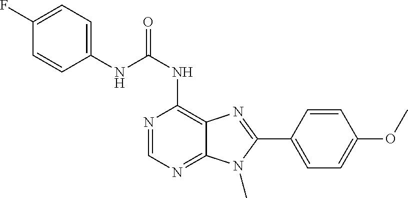 [SMILES: COc1ccc(-c2nc3c(NC(=O)Nc4ccc(F)cc4)ncnc3n2C)cc1] |
| 4 | 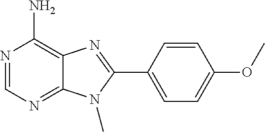 [SMILES: COc1ccc(-c2nc3c(N)ncnc3n2C)cc1] (4) | 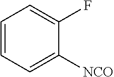 | 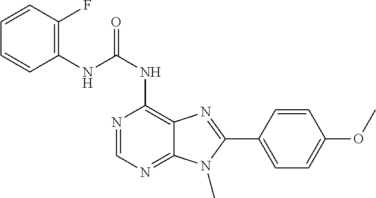 [SMILES: COc1ccc(-c2nc3c(NC(=O)Nc4ccccc4F)ncnc3n2C)cc1] |
| 5 | 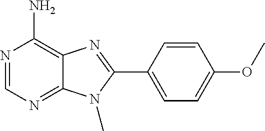 [SMILES: COc1ccc(-c2nc3c(N)ncnc3n2C)cc1] (4) | 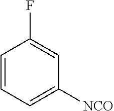 | 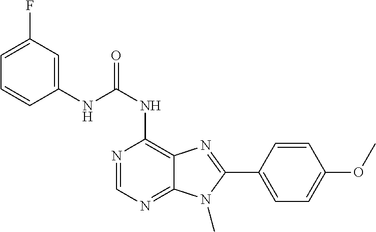 [SMILES: COc1ccc(-c2nc3c(NC(=O)Nc4cccc(F)c4)ncnc3n2C)cc1] |
| 6 | 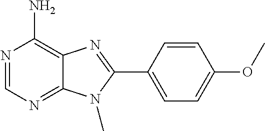 [SMILES: COc1ccc(-c2nc3c(N)ncnc3n2C)cc1] (4) | 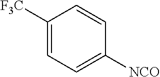 | 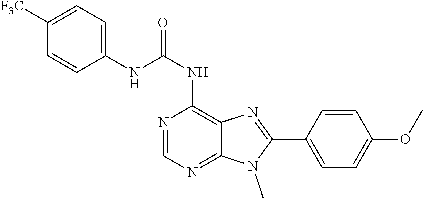 [SMILES: COc1ccc(-c2nc3c(NC(=O)Nc4ccc(C(F)(F)F)cc4)ncnc3n2C)cc1] |

Table 4 provide the structure of reactants and products obtained with reaction using sulphonyl chloride and NaH

TABLE 4

| Serial No | Amine (A) | 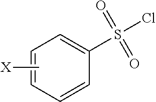 | B (Sulphonamide compounds) |
|---|---|---|---|
| 1 | 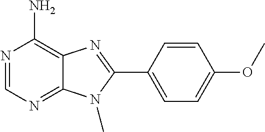 [SMILES: COc1ccc(-c2nc3c(N)ncnc3n2C)cc1] (4) | 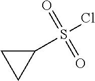 | 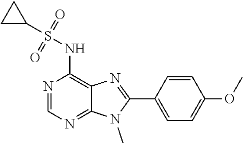 [SMILES: COc1ccc(-c2nc3c(NS(=O)(=O)C4CC4)ncnc3n2C)cc1] |
| 2 | 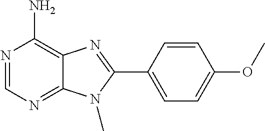 [SMILES: COc1ccc(-c2nc3c(N)ncnc3n2C)cc1] (4) | 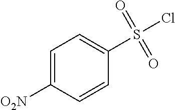 [SMILES: O=[N+]([O-])c1ccc(S(=O)(=O)Cl)cc1] | 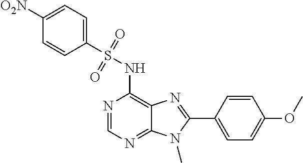 [SMILES: COc1ccc(-c2nc3c(NS(=O)(=O)c4ccc([N+](=O)[O-])cc4)ncnc3n2C)cc1] |
| 3 | 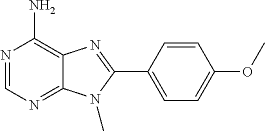 [SMILES: COc1ccc(-c2nc3c(N)ncnc3n2C)cc1] (4) | 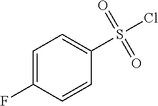 [SMILES: O=S(=O)(Cl)c1ccc(F)cc1] | 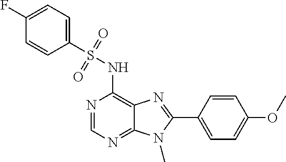 [SMILES: COc1ccc(-c2nc3c(NS(=O)(=O)c4ccc(F)cc4)ncnc3n2C)cc1] |
| 4 | 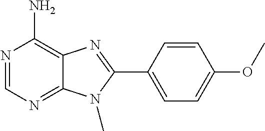 [SMILES: COc1ccc(-c2nc3c(N)ncnc3n2C)cc1] (4) | 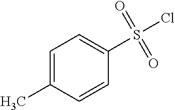 | 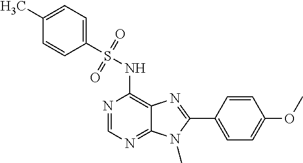 |
| 5 | 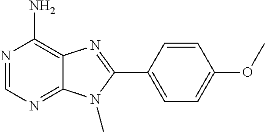 [SMILES: COc1ccc(-c2nc3c(N)ncnc3n2C)cc1] (4) | 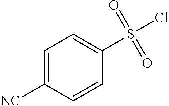 [SMILES: N#Cc1ccc(S(=O)(=O)Cl)cc1] | 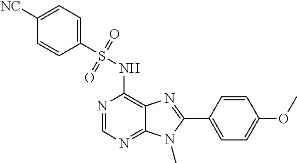 [SMILES: COc1ccc(-c2nc3c(NS(=O)(=O)c4ccc(C#N)cc4)ncnc3n2C)cc1] |
| 6 | 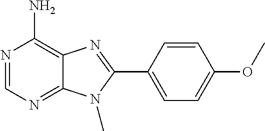 [SMILES: COc1ccc(-c2nc3c(N)ncnc3n2C)cc1] (4) | 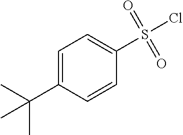 | 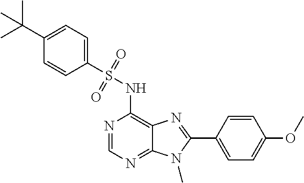 [SMILES: COc1ccc(-c2nc3c(NS(=O)(=O)c4ccc(C(C)(C)C)cc4)ncnc3n2C)cc1] |
| 7 | 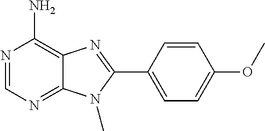 [SMILES: COc1ccc(-c2nc3c(N)ncnc3n2C)cc1] (4) | 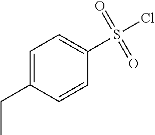 | 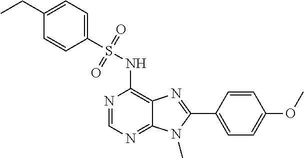 [SMILES: CCc1ccc(S(=O)(=O)Nc2ncnc3c2nc(-c2ccc(OC)cc2)n3C)cc1] |
| 8 | 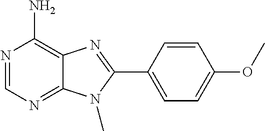 [SMILES: COc1ccc(-c2nc3c(N)ncnc3n2C)cc1] (4) | 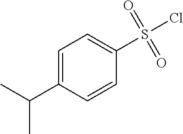 | 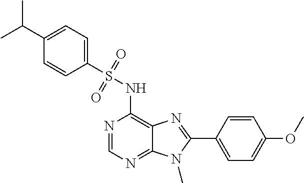 [SMILES: COc1ccc(-c2nc3c(NS(=O)(=O)c4ccc(C(C)C)cc4)ncnc3n2C)cc1] |
| 9 | 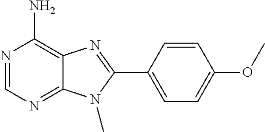 [SMILES: COc1ccc(-c2nc3c(N)ncnc3n2C)cc1] (4) | 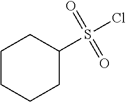 | 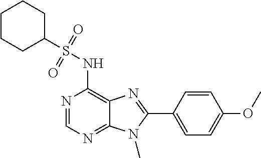 [SMILES: COc1ccc(-c2nc3c(NS(=O)(=O)C4CCCCC4)ncnc3n2C)cc1] |
| 10 | 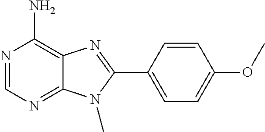 [SMILES: COc1ccc(-c2nc3c(N)ncnc3n2C)cc1] (4) | 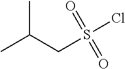 | 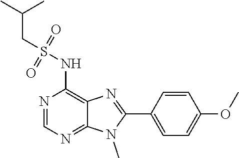 [SMILES: COc1ccc(-c2nc3c(NS(=O)(=O)CC(C)C)ncnc3n2C)cc1] |
| 11 | 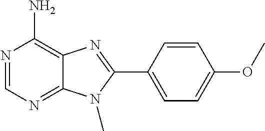 [SMILES: COc1ccc(-c2nc3c(N)ncnc3n2C)cc1] (4) | 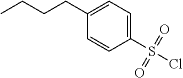 | 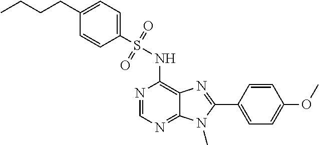 [SMILES: CCCCc1ccc(S(=O)(=O)Nc2ncnc3c2nc(-c2ccc(OC)cc2)n3C)cc1] |
| 12 | 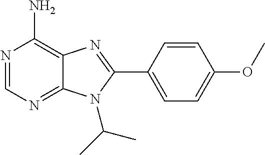 [SMILES: COc1ccc(-c2nc3c(N)ncnc3n2C(C)C)cc1] (29) | 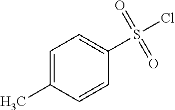 | 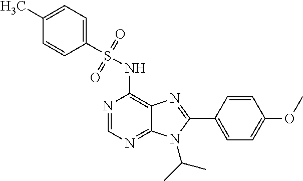 |
| 13 | 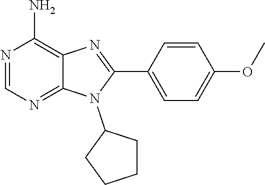 [SMILES: COc1ccc(-c2nc3c(N)ncnc3n2C2CCCC2)cc1] (31) | 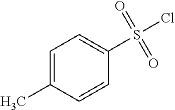 | 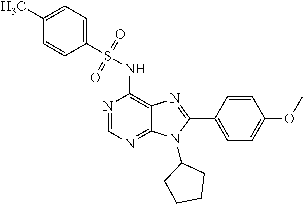 |
| 14 | 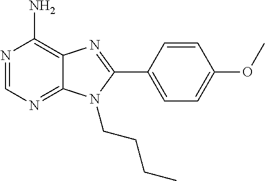 [SMILES: CCCCn1c(-c2ccc(OC)cc2)nc2c(N)ncnc21] (33) | 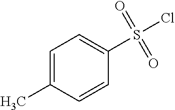 | 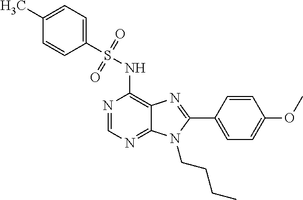 |
| 15 | 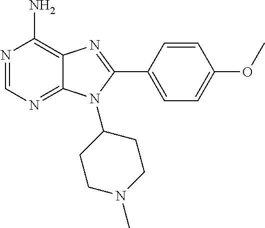 [SMILES: COc1ccc(-c2nc3c(N)ncnc3n2C2CCN(C)CC2)cc1] (35) | 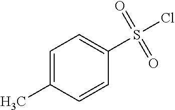 | 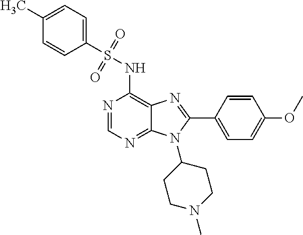 |
| 16 | 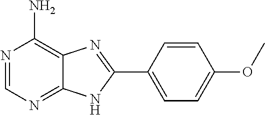 [SMILES: COc1ccc(-c2nc3c(N)ncnc3[nH]2)cc1] (28) | 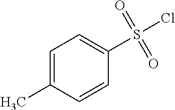 | 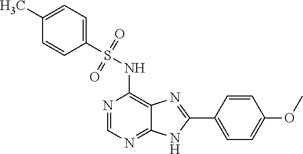 |
| 17 | 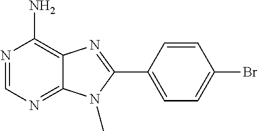 [SMILES: Cn1c(-c2ccc(Br)cc2)nc2c(N)ncnc21] (38) | 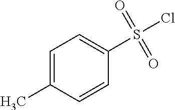 | 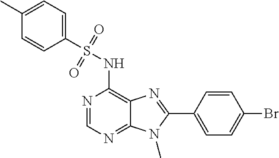 [SMILES: Cc1ccc(S(=O)(=O)Nc2ncnc3c2nc(-c2ccc(Br)cc2)n3C)cc1] |
| 18 | 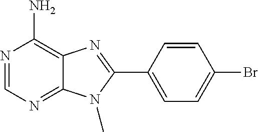 [SMILES: Cn1c(-c2ccc(Br)cc2)nc2c(N)ncnc21] (38) | 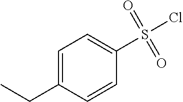 | 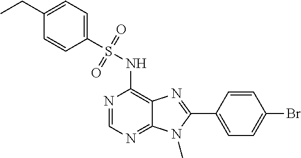 [SMILES: CCc1ccc(S(=O)(=O)Nc2ncnc3c2nc(-c2ccc(Br)cc2)n3C)cc1] |
| 19 | 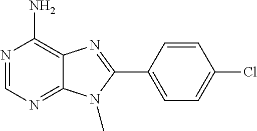 [SMILES: Cn1c(-c2ccc(Cl)cc2)nc2c(N)ncnc21] (41) | 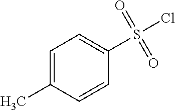 | 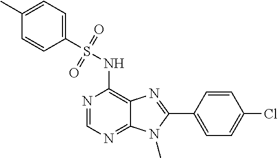 [SMILES: Cc1ccc(S(=O)(=O)Nc2ncnc3c2nc(-c2ccc(Cl)cc2)n3C)cc1] |
| 20 | 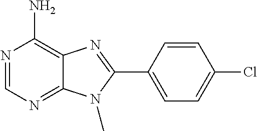 [SMILES: Cn1c(-c2ccc(Cl)cc2)nc2c(N)ncnc21] (41) | 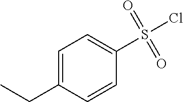 | 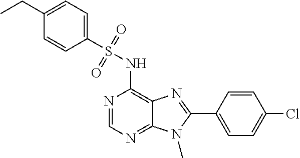 [SMILES: CCc1ccc(S(=O)(=O)Nc2ncnc3c2nc(-c2ccc(Cl)cc2)n3C)cc1] |
| 21 | 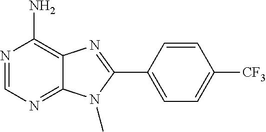 [SMILES: Cn1c(-c2ccc(C(F)(F)F)cc2)nc2c(N)ncnc21] (44) | 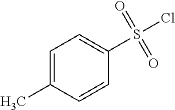 | 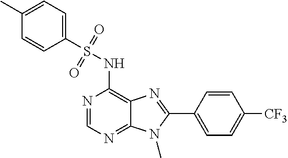 [SMILES: Cc1ccc(S(=O)(=O)Nc2ncnc3c2nc(-c2ccc(C(F)(F)F)cc2)n3C)cc1] |
| 22 | 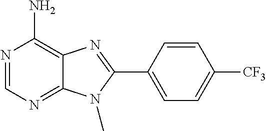 [SMILES: Cn1c(-c2ccc(C(F)(F)F)cc2)nc2c(N)ncnc21] (44) | 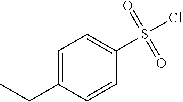 | 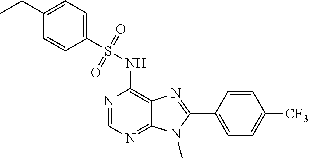 [SMILES: CCc1ccc(S(=O)(=O)Nc2ncnc3c2nc(-c2ccc(C(F)(F)F)cc2)n3C)cc1] |
| 23 | 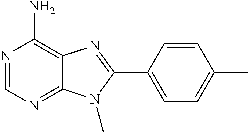 [SMILES: Cc1ccc(-c2nc3c(N)ncnc3n2C)cc1] (47) | 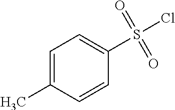 | 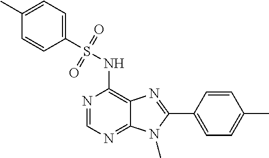 |
| 24 | 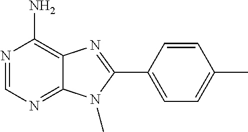 [SMILES: Cc1ccc(-c2nc3c(N)ncnc3n2C)cc1] (47) | 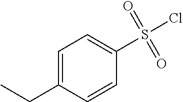 | 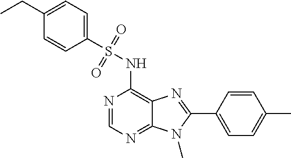 [SMILES: CCc1ccc(S(=O)(=O)Nc2ncnc3c2nc(-c2ccc(C)cc2)n3C)cc1] |
| 25 | 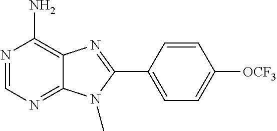 [SMILES: Cn1c(-c2ccc(OC(F)(F)F)cc2)nc2c(N)ncnc21] (50) | 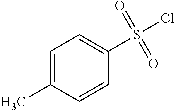 | 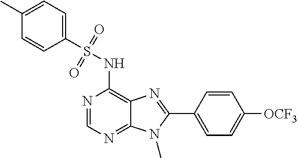 [SMILES: Cc1ccc(S(=O)(=O)Nc2ncnc3c2nc(-c2ccc(OC(F)(F)F)cc2)n3C)cc1] |
| 26 | 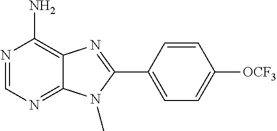 [SMILES: Cn1c(-c2ccc(OC(F)(F)F)cc2)nc2c(N)ncnc21] (50) | 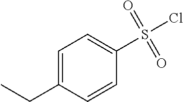 | 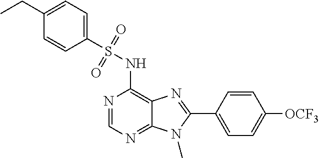 [SMILES: CCc1ccc(S(=O)(=O)Nc2ncnc3c2nc(-c2ccc(OC(F)(F)F)cc2)n3C)cc1] |
| 27 | 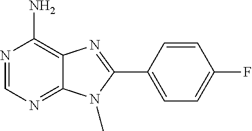 [SMILES: Cn1c(-c2ccc(F)cc2)nc2c(N)ncnc21] (38) | 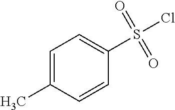 | 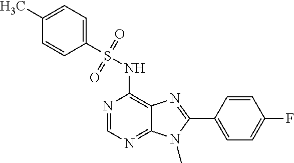 [SMILES: Cc1ccc(S(=O)(=O)Nc2ncnc3c2nc(-c2ccc(F)cc2)n3C)cc1] |
| 28 | 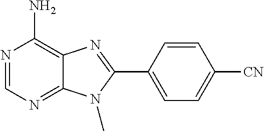 [SMILES: Cn1c(-c2ccc(C#N)cc2)nc2c(N)ncnc21] (42) | 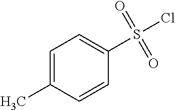 | 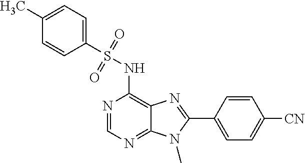 [SMILES: Cc1ccc(S(=O)(=O)Nc2ncnc3c2nc(-c2ccc(C#N)cc2)n3C)cc1] |
| 29 | 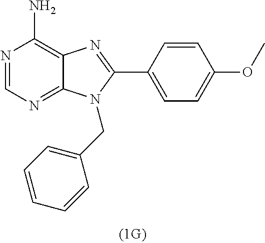 [SMILES: COc1ccc(-c2nc3c(N)ncnc3n2Cc2ccccc2)cc1] | 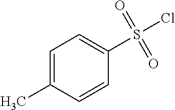 | 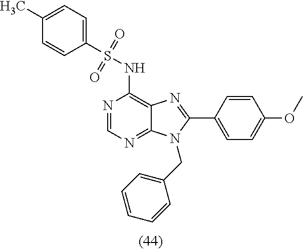 |
| 30 | 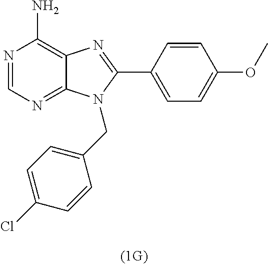 [SMILES: COc1ccc(-c2nc3c(N)ncnc3n2Cc2ccc(Cl)cc2)cc1] | 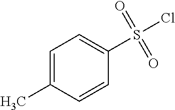 | 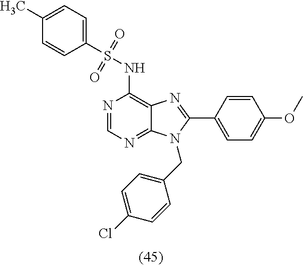 |

### Abbreviations

NaOH Sodium Hydroxide
TBAF Tetrabutylammonium fluoride
Mel Methyl Iodide
MeOH Methanol
CHCl₃ Chloroform
d₆-DMSO Dimethyl sulfoxide-d6
NaOAc Sodium Acetate
Na₂S₂O₃ Sodium Thiosulphate
EtOAc Ethyl Acetate
K₂CO₃ Potassium Carbonate
Pd(PPh3)4 Tetrakis(triphenylphosphine)palladium(0)
THF Tetrahydrofuran
LiHMDS Lithium bis(trimethylsilyl)amide
NaH Sodium Hydride

Still another embodiment of the present application provides a compound having structure I or salts thereof which can be used to augment the effector function (IFNγ production) of T cells and hence heightened their anti-tumor response.

Another embodiment of the present invention provides a compound having structure I or salts thereof to establish the Structure-Activity-Relationship (SAR) among the small molecules mTOR activator.

Yet another embodiment of the present invention provides a compound having structure I or salts thereof to evaluate the potential of small molecule activator of mTOR in improving the metabolic fitness of T cells.

Another embodiment of the present invention provides a compound having structure I or salts thereof along with pharmaceutically acceptable excipients.

Still another embodiment of the present invention provides a method to evaluate the potential of mTOR activator in modulating the expression of immune checkpoint inhibitors (PD1, Tim3, Lag3, CD38) on T cells.

Yet another aspect of the present invention provides a method to evaluate the efficacy of mTOR activator-induced anti-tumor T cells in generating long-lasting anti-tumor response in tumor bearing mice.

### EXAMPLES

Following examples are given by way of illustration and therefore should not be construed to limit the scope of the present invention.

Temperatures are given in degree Celsius. The structures of final products, intermediates and starting materials are confirmed by standard analytical methods, spectroscopic characterization e.g., MS, NMR. Abbreviations used are those conventional in the art.

All starting materials, reagents, catalysts, building blocks, acids, bases, dehydrating agents and solvents utilized to synthesize the compounds of the present invention are either commercially available or can be produced by known organic synthesis methods in the art.

### Example 1

### General Procedure A: Amide Formation Reaction

Compound 4 (1 equivalent) was dissolved in dry THF (5-10 mL) and then LiHMDS (2 equivalent) was added dropwise at 0° C. temperature keeping nitrogen atmosphere and the reaction mixture was allowed to stir at room temperature for 15 minutes. Then a solution of required acid chlorides (1.2 equivalents) in dry THF (1-2 mL) was added dropwise at 0° C. temperature and then stirred at room temperature for another 30 minutes. After completion, reaction mass was washed with water and extracted with ethyl acetate and evaporated. If needed column chromatography was performed to get the pure product.

### Example 2

### General Procedure B: Urea Formation Reaction

Compound 4 (1 equivalent) was dissolved in dry THF (5-10 mL) and then LiHMDS (2 equivalent) was added dropwise at 0° C. temperature keeping nitrogen atmosphere and the reaction mixture was allowed to stir at room temperature for 15 minutes. Then a solution of required isocyanate (1.2 equivalents) in dry THF was added dropwise at 0° C. temperature and then stirred at room temperature for another 30 minutes. After completion, workup was done with water and ethyl acetate and then organic layer was evaporated. If needed column chromatography was performed to get the pure product.

### Example 3

### General Procedure C: Sulphonamide Formation Reaction

Compound 4, 38, 40, 42 (1 equivalent) was dissolved in dry DMF (5 mL) and then NaH (2 equivalent) was added portion wise at 0° C. temperature keeping nitrogen atmosphere and the reaction mixture was allowed to stir at room temperature for 15 minutes. Then required sulfonyl chlorides (1.2 equivalents) were added portion wise at 0° C. temperature and then stirred at room temperature for another 30 minutes. After completion; reaction mixture was washed with ice cold water and extracted with EtOAc. If needed column chromatography was performed to get the pure product.

### Example 4

### Preparation of 9-methyl-9H-purin-6-amine (2)

Adenine 1 (5 gm, 37.03 mmole) was taken in (1:1) Dioxane and water (30 mL) mixture and NaOH (1.48 gm, 37.03) was added. Then TBAF (37.03 mL, 37.03 mmole) was added in the reaction mixture. Next Mel (1.8 mL, 55.54 mmole) was added dropwise at 0° C. temperature and the reaction mixture were allowed to stir at room temperature for overnight. After completion of the reaction solvent was evaporated in reduced pressure and the product was purified by column chromatography (Silica gel, mesh size 100-200) eluting (5% MeOH/CHCl₃) to obtain compound 2 (3.9 gm, 72%) as off white solid. ¹H NMR (400 MHz, d₆-DMSO) δ in ppm 8.10 (s, 1H), 8.02 (s, 1H), 7.06 (s, 2H), 3.67 (s, 3H).

### Example 5

### Preparation of 8-bromo-9-methyl-9H-purin-6-amine (3)

Compound 2 (1 gm, 6.70 mmole) was dissolved in a (9:1) mixture of Dioxane and 0.5(M) NaOAc solution (10 mL). Then liq.bromine (0.4 mL, 10.05 mmole) was added dropwise in reaction mixture and stirred at room temperature for overnight. After completion of the reaction mixture was washed with satd. solution of Na₂S₂O₃ and extracted with EtOAc. The product was then purified by column chromatography (Silica gel, mesh size 100-200) eluting (5% MeOH/CHCl₃) to obtain 3 as pale yellow amorphous solid (0.944 g, 62%). ¹H NMR (400 MHz, d₆-DMSO) δ in ppm 8.08 (s, 1H), 7.30 (s, 2H), 3.60 (s, 3H).

### Example 6

### Preparation of 8-(4-methoxyphenyl)-9-methyl-9H-purin-6-amine (4)

Compound 3 (0.5 gm, 1.95 mmole), 4-methoxyphenylboronicacid (0.446 gm, 2.92 mmole), K₂CO₃ (0.541 gm, 3.9 mmole) were taken in a pressure tube and dissolved in (9:1) mixture of Dioxane& water (10 mL). Solution was purged with Argon gas for 30 minutes. Then catalyst Pd(PPh₃)₄ (0.226 gm, 0.19 mmole) was added and the reaction mixture was stirred at 100° C.-110° C. for overnight. Reaction was monitored by checking TLC. After completion, reaction mass was washed with water and extracted with ethyl acetate and evaporated. The product was then purified by column chromatography (Silica gel, mesh size 100-200) eluting (3% MeOH/CHCl₃) to obtain 4 as pale yellow amorphous solid (0.421 g, 75%). ¹H NMR (400 MHz, d₆-DMSO) δ in ppm 8.12 (s, 1H), 7.77-7.75 (m, 2H), 7.20 (s, 2H), 7.08-7.05 (m, 2H).

### Example 7

### Preparation of N-(8-(4-methoxyphenyl)-9-methyl-9H-purin-6-yl)-4-nitrobenzamide (5)

The following compound was made by general procedure A using 4 (0.10 g, 0.39 mmol), dry THF (5 mL), LiHMDS (0.78 mL, 0.78 mmol), 4-nitrobenzoyl chloride (0.087 g, 0.46 mmol) to obtain compound 5 (0.087 g, 55%) as off white solid. ¹H NMR (400 MHz, d₆-DMSO) δ in ppm 8.78 (s, 1H), 8.32-8.28 (m, 2H), 8.26-8.22 (m, 2H), 7.70-7.68 (m, 2H), 7.06-7.04 (m, 2H), 3.92 (s, 3H), 3.86 (s, 3H).

### Example 8

### Preparation of 4-fluoro-N-(8-(4-methoxyphenyl)-9-methyl-9H-purin-6-yl)benzamide (6)

The following compound was made by general procedure A using 4 (0.10 g, 0.39 mmol), dry THF (5 mL), LiHMDS (0.78 mL, 0.78 mmol), 4-fluorobenzoyl chloride (0.056 mL, 0.46 mmol) to obtain compound 6 (0.062 g, 42%) as off white solid. ¹H NMR (400 MHz, CDCl₃) δ in ppm 9.26 (s, 1H), 8.81 (s, 1H), 8.07-8.03 (m, 2H), 7.76-7.73 (m, 2H), 7.18-7.14 (m, 2H), 7.07-7.04 (m, 2H), 3.93 (s, 3H), 3.88 (s, 3H).

### Example 9

### Preparation of 4-methoxy-N-(8-(4-methoxyphenyl)-9-methyl-9H-purin-6-yl)benzamide (7)

The following compound was made by general procedure A using 4 (0.10 g, 0.39 mmol), dry THF (5 mL), LiHMDS (0.78 mL, 0.78 mmol), 4-methoxybenzoyl chloride (0.063 mL, 0.46 mmol) to obtain compound 7 (0.061 g, 40%) as off white solid. ¹H NMR (400 MHz, d₆-DMSO) δ in ppm 10.92 (s, 1H), 8.67 (s, 1H), 8.04-7.96 (m, 2H), 7.85-7.79 (m, 2H), 7.11-7.07 (m, 2H), 7.05-7.00 (m, 2H), 3.86 (s, 3H), 3.82 (s, 3H), 3.81 (s, 3H).

### Example 10

### Preparation of N-(8-(4-methoxyphenyl)-9-methyl-9H-purin-6-yl)benzamide (8)

The following compound was made by general procedure A using 4 (0.10 g, 0.39 mmol), dry THF (5 mL), LiHMDS (0.78 mL, 0.78 mmol), benzoyl chloride (0.054 mL, 0.46 mmol) to obtain compound 8 (0.084 g, 60%) was off white solid. ¹H NMR (400 MHz, CDCl₃) δ in ppm 9.27 (s, 1H), 8.82 (s, 1H), 8.05-7.99 (m, 2H), 7.77-7.73 (m, 2H), 7.60-7.55 (m, 2H), 7.52-7.46 (m, 2H), 7.08-7.03 (m, 2H), 3.94 (s, 3H), 3.88 (s, 3H).

### Example 11

### Preparation of N-(8-(4-methoxyphenyl)-9-methyl-9H-purin-6-yl)-4-(trifluoromethyl)benzamide (9)

The following compound was made by general procedure A using 4 (0.10 g, 0.39 mmol), dry THF (5 mL), LiHMDS (0.78 mL, 0.78 mmol), 4-(trifluoromethyl)benzoyl chloride (0.070 mL, 0.46 mmol) to obtain compound 9 (0.058 g, 35%) as off white solid. ¹H NMR (400 MHz, CDCl₃) δ in ppm 9.46 (s, 1H), 8.80 (s, 1H), 8.20-8.10 (m, 2H), 7.76-7.71 (m, 4H), 7.07-7.03 (m, 2H), 3.94 (s, 3H), 3.88 (s, 3H).

### Example 12

### Preparation of 1-cyclopentyl-3-(8-(4-methoxyphenyl)-9-methyl-9H-purin-6-yl)urea (10)

The following compound was made by general procedure B using 4 (0.10 g, 0.39 mmol), dry THF (5 mL), LiHMDS (0.78 mL, 0.78 mmol), isocyanatocyclopentane (0.053 mL, 0.46 mmol) to obtain compound 10 (0.074 g, 52%) as off white solid. ¹H NMR (400 MHz, CDCl₃) δ in ppm 9.45 (d, J=7.2 Hz, 1H), 8.49 (s, 1H), 7.90 (s, 1H), 7.73-7.69 (m, 2H), 7.06-7.02 (m, 2H), 4.29-4.21 (m, 1H), 3.90 (s, 3H), 3.88 (s, 3H), 2.08-2.00 (m, 2H), 1.79-1.73 (m, 2H), 1.68-1.57 (m, 4H).

### Example 13

### Preparation of 1-(8-(4-methoxyphenyl)-9-methyl-9H-purin-6-yl)-3-phenylurea (11)

The following compound was made by general procedure B using 4 (0.10 g, 0.39 mmol), dry THF (5 mL), LiHMDS (0.78 mL, 0.78 mmol), Phenyl isocyanate (0.051 mL, 0.46 mmol) to obtain compound 11 (0.095 g, 65%) as off white solid. ¹H NMR (400 MHz, CDCl₃) δ in ppm 11.78 (s, 1H), 8.60 (s, 1H), 7.76-7.72 (m, 2H), 7.65-7.61 (m, 2H), 7.36-7.31 (m, 2H), 7.11-7.08 (m, 1H), 7.08-7.04 (m, 2H), 3.92 (s, 3H), 3.88 (s, 3H).

### Example 14

### Preparation of 1-(4-fluorophenyl)-3-(8-(4-methoxyphenyl)-9-methyl-9H-purin-6-yl)urea (12)

The following compound was made by general procedure B using 4 (0.10 g, 0.39 mmol), dry THF (5 mL), LiHMDS (0.78 mL, 0.78 mmol), 4-Fluorophenyl isocyanate (0.053 mL, 0.46 mmol) to obtain compound 12 (0.064 g, 42%) as off white solid. ¹H NMR (400 MHz, d₆-DMSO) δ in ppm 11.83 (s, 1H), 9.99 (s, 1H), 8.63 (s, 1H), 7.88-7.84 (m, 2H), 7.63-7.59 (m, 2H), 7.18-7.11 (m, 4H), 3.85 (s, 3H), 3.83 (s, 3H).

### Example 15

### Preparation of 1-(2-fluorophenyl)-3-(8-(4-methoxyphenyl)-9-methyl-9H-purin-6-yl)urea (13)

The following compound was made by general procedure B using 4 (0.10 g, 0.39 mmol), dry THF (5 mL), LiHMDS (0.78 mL, 0.78 mmol), 2-Fluorophenyl isocyanate (0.053 mL, 0.46 mmol) to obtain compound 13 (0.064 g, 42%) as off white solid. ¹H NMR (400 MHz, CDCl₃) δ in ppm 12.14 (s, 1H), 8.62 (s, 1H), 8.37-8.30 (m, 2H), 8.18 (s, 1H), 7.76-7.71 (m, 2H), 7.15-7.09 (m, 2H), 7.07-7.03 (m, 2H), 7.03-6.99 (m, 1H), 3.93 (s, 3H), 3.88 (s, 3H).

### Example 16

Preparation of 1-(3-fluorophenyl)-3-(8-(4-methoxyphenyl)-9-methyl-9H-purin-6-yl)urea (14): The following compound was made by general procedure B using 4 (0.10 g, 0.39 mmol), dry THF (5 mL), LiHMDS (0.78 mL, 0.78 mmol), 3-Fluorophenyl isocyanate (0.053 mL, 0.46 mmol) to obtain compound 14 (0.060 g, 40%) as off white solid. ¹H NMR (400 MHz, CDCl₃) δ in ppm 11.94 (s, 1H), 8.61 (s, 1H), 8.11 (s, 1H), 7.76-7.73 (m, 2H), 7.59-7.54 (m, 1H), 7.31-7.21 (m, 2H), 7.09-7.05 (m, 2H), 6.81-6.86 (m, 1H), 3.94 (s, 3H), 3.89 (s, 3H).

### Example 17

### Preparation of 1-(8-(4-methoxyphenyl)-9-methyl-9H-purin-6-yl)-3-(4-(trifluoromethyl)phenyl)urea (15)

The following compound was made by general procedure B using 4 (0.10 g, 0.39 mmol), dry THF (5 mL), LiHMDS (0.78 mL, 0.78 mmol), 4-(Trifluoromethyl)phenyl isocyanate (0.056 mL, 0.46 mmol) to obtain compound 15 (0.068 g, 35%) as off white solid. 1H NMR (400 MHz, CDCl₃) δ in ppm 8.55 (s, 1H), 7.69-7.63 (m, 2H), 7.53-7.48 (m, 2H), 7.03-6.98 (m, 2H), 3.86 (s, 3H), 3.82 (s, 3H).

### Example 18

### Preparation of N-(8-(4-methoxyphenyl)-9-methyl-9H-purin-6-yl)cyclopropanesulfonamide (16)

The following compound was made by general procedure D using 4 (0.10 g, 0.39 mmol), dry DMF (5 mL), NaH (18.81 mg, 0.78 mmol), cyclopropanesulfonyl chloride (0.056 mL, 0.46 mmol) to obtain compound 16 (0.045 g, 32%) as off white solid. ¹H NMR (400 MHz, CDCl₃) δ in ppm 8.27 (s, 1H), 7.59-7.52 (m, 2H), 6.96-6.89 (m, 2H), 3.75 (s, 3H), 3.74 (s, 3H), 3.20-3.16 (m, 1H), 1.27-1.15 (m, 2H), 0.96-0.87 (m, 2H).

### Example 19

### Preparation of N-(8-(4-methoxyphenyl)-9-methyl-9H-purin-6-yl)-4-nitrobenzenesulfonamide (17)

The following compound was made by general procedure D using 4 (0.10 g, 0.39 mmol), dry DMF (5 mL), NaH (18.81 mg, 0.78 mmol), 4-nitrobenzene-1-sulfonyl chloride (0.104 gm, 0.46 mmol) to obtain compound 17 (0.064 g, 33%) as off white solid. 1H NMR (400 MHz, d₆-DMSO) δ in ppm 8.36 (s, 1H), 8.34-8.30 (m, 2H), 8.19-8.13 (m, 2H), 7.75-7.70 (m, 2H), 7.11-7.05 (m, 2H), 3.81 (s, 3H), 3.78 (s, 3H).

### Example 20

### Preparation of 4-fluoro-N-(8-(4-methoxyphenyl)-9-methyl-9H-purin-6-yl)benzenesulfonamide (18)

The following compound was made by general procedure D using 4 (0.10 g, 0.39 mmol), dry DMF (5 mL), NaH (18.81 mg, 0.78 mmol), 4-fluorobenzene-1-sulfonyl chloride (0.091 gm, 0.46 mmol) to obtain compound 18 (0.064 g, 40%) as off white solid.

### Example 21

### Preparation of N-(8-(4-methoxyphenyl)-9-methyl-9H-purin-6-yl)-4-methylbenzenesulfonamide (19)

The following compound was made by general procedure D using 4 (0.10 g, 0.39 mmol), dry DMF (5 mL), NaH (18.81 mg, 0.78 mmol), 4-methylbenzene-1-sulfonyl chloride (0.089 gm, 0.46 mmol) to obtain compound 19 (0.048 g, 30%) as off white solid. ¹H NMR (400 MHz, d₆-DMSO) δ in ppm 8.32 (s, 1H), 7.88-7.27 (m, 4H), 7.36-7.29 (m, 2H), 7.13-7.06 (m, 2H), 3.81 (m, 1H), 3.78 (m, 1H), 2.31 (s, 3H).

### Example 22

### Preparation of 4-cyano-N-(8-(4-methoxyphenyl)-9-methyl-9H-purin-6-yl)benzenesulfonamide (20)

The following compound was made by general procedure D using 4 (0.10 g, 0.39 mmol), dry DMF (5 mL), NaH (18.81 mg, 0.78 mmol), 4-cyanobenzene-1-sulfonyl chloride (0.094 g, 0.46 mmol) to obtain compound 20 (0.052 g, 32%) as off white solid.

### Example 23

### Preparation of 4-(tert-butyl)-N-(8-(4-methoxyphenyl)-9-methyl-9H-purin-6-yl)benzenesulfonamide (21)

The following compound was made by general procedure D using 4 (0.10 g, 0.39 mmol), dry DMF (5 mL), NaH (18.81 mg, 0.78 mmol), 4-(tert-butyl)benzene-1-sulfonyl chloride (0.109 g, 0.46 mmol) to obtain compound 21 (0.063 g, 36%) as off white solid. ¹H NMR (400 MHz, CDCl₃) δ in ppm 8.29 (br.s, 1H), 8.05-7.97 (m, 2H), 7.71-7.67 (m, 2H), 7.46-7.43 (m, 2H), 7.02-6.98 (m, 2H), 3.85 (s, 6H), 1.28 (s, 9H).

### Example 24

### Preparation of 4-ethyl-N-(8-(4-methoxyphenyl)-9-methyl-9H-purin-6-yl)benzenesulfonamide (22)

The following compound was made by general procedure D using 4 (0.10 g, 0.39 mmol), dry DMF (5 mL), NaH (18.81 mg, 0.78 mmol), 4-ethylbenzene-1-sulfonyl chloride (0.096 g, 0.46 mmol) to obtain compound 22 (0.069 g, 42%) as off white solid.

### Example 25

### Preparation of 4-isopropyl-N-(8-(4-methoxyphenyl)-9-methyl-9H-purin-6-yl)benzenesulfonamide (23)

The following compound was made by general procedure D using 4 (0.10 g, 0.39 mmol), dry DMF (5 mL), NaH (18.81 mg, 0.78 mmol), 4-isopropylbenzene-1-sulfonyl chloride (0.102 g, 0.46 mmol) to obtain compound 23 (0.061 g, 36%) as off white solid. 1H NMR (400 MHz, CDCl₃) δ in ppm 8.27 (br.s, 1H), 8.04-7.97 (m, 2H), 7.70-7.67 (m, 2H), 7.30-7.26 (m, 2H), 7.01-6.98 (m, 2H), 3.86 (s, 3H), 3.85 (s, 3H), 2.96-2.86 (m, 1H), 1.21 (d, J=7.2 Hz, 6H).

### Example 26

### Preparation of N-(8-(4-methoxyphenyl)-9-methyl-9H-purin-6-yl)cyclohexanesulfonamide (24)

The following compound was made by general procedure D using 4 (0.10 g, 0.39 mmol), dry DMF (5 mL), NaH (18.81 mg, 0.78 mmol), cyclohexanesulfonyl chloride (0.085 g, 0.46 mmol) to obtain compound 24 (0.070 g, 45%) as off white solid.

### Example 27

### Preparation of N-(8-(4-methoxyphenyl)-9-methyl-9H-purin-6-yl)-2-methylpropane-1-sulfonamide (25)

The following compound was made by general procedure D using 4 (0.10 g, 0.39 mmol), dry DMF (5 mL), NaH (18.81 mg, 0.78 mmol), 2-methylpropane-1-sulfonyl chloride (0.073 g, 0.46 mmol) to obtain compound 25 (0.044 g, 30%) as off white solid.

### Example 28

### Preparation of 4-butyl-N-(8-(4-methoxyphenyl)-9-methyl-9H-purin-6-yl)benzenesulfonamide (26)

The following compound was made by general procedure D using 4 (0.10 g, 0.39 mmol), dry DMF (5 mL), NaH (18.81 mg, 0.78 mmol), 4-butylbenzene-1-sulfonyl chloride (0.109 g, 0.46 mmol) to obtain compound 26 (0.047 g, 27%) as off white solid. 1H NMR (400 MHz, CDCl₃) δ in ppm 8.27 (s, 1H), 8.04-7.94 (m, 2H), 7.72-7.67 (m, 2H), 7.26-7.22 (m, 2H), 7.03-6.98 (m, 2H), 3.86 (s, 3H), 3.85 (s, 3H), 2.61 (t, J=7.6 Hz, 2H), 1.59-1.51 (m, 2H), 1.35-1.26 (m, 2H), 0.88 (t, J=7.32 Hz, 3H).

### Example 29

### Preparation of 8-bromo-9H-purin-6-amine (27)

Compound 1 (1 gm, 7.40 mmole) was dissolved in a (9:1) mixture of Dioxane and 0.5(M) NaOAc solution (10 mL). Then liq.bromine (0.44 mL, 11.10 mmole) was added dropwise in reaction mixture and stirred at room temperature for overnight. After completion of the reaction mixture was washed with satd solution of Na₂S₂O₃ and extracted with EtOAc.

### Example 30

### Preparation of 8-(4-methoxyphenyl)-9H-purin-6-amine (28)

Compound 27 (0.5 gm, 2.34 mmol), 4-methoxyphenylboronicacid (0.533 gm, 3.51 mmol), K2CO3 (0.648 gm, 4.68 mmol) were taken in a pressure tube and dissolved in (9:1) mixture of Dioxane& water (10 mL). Solution was purged with Argon gas for 30 minutes. Then catalyst Pd(PPh₃)₄ (0.271 gm, 0.23 mmol) was added and the reaction mixture was stirred at 100° C.-110° C. for overnight. Reaction was monitored by checking TLC.

### Example 31

### Preparation of 9-isopropyl-8-(4-methoxyphenyl)-9H-purin-6-amine (29)

To a suspension of 28 (0.5 gm, 2.07 mmol) in 8 mL of DMF was added K₂CO₃ (0.585 gm, 4.24 mmol). The reaction mixture was stirred at 60° C. for 30 min. A solution of isopropyl iodide (0.3 mL, 3.10 mmol) in 3 mL DMF was added to the reaction mixture and the mixture was stirred at 65° C. for 72 hr.

### Example 32

### Preparation of N-(9-isopropyl-8-(4-methoxyphenyl)-9H-purin-6-yl)-4-methylbenzenesulfonamide (30)

The following compound was made by general procedure D using 29 (0.10 g, 0.35 mmol), dry DMF (5 mL), NaH (16.80 mg, 0.70 mmol), 4-methylbenzene-1-sulfonyl chloride (0.099 gm, 0.52 mmol) to obtain compound 30. 1H NMR (400 MHz, Chloroform-d) δ 8.25 (s, 1H), 7.98 (d, J=3.6 Hz, 2H), 7.52 (d, J=8.8 Hz, 2H), 7.23 (d, J=8.0 Hz, 2H), 7.01 (d, J=8.8 Hz, 2H), 4.71 (p, J=6.8 Hz, 1H), 3.86 (s, 3H), 2.36 (s, 3H), 1.67 (s, 3H), 1.65 (s, 3H).

### Example 33

### Preparation of 9-cyclopentyl-8-(4-methoxyphenyl)-9H-purin-6-amine (31)

To a suspension of 28 (0.5 gm, 2.07 mmol) in 8 mL of DMF was added K₂CO₃ (0.572 gm, 4.14 mmol). The reaction mixture was stirred at 60° C. for 30 min. A solution of bromocyclopentane (0.33 mL, 3.10 mmol) in 3 mL DMF was added to the reaction mixture and the mixture was stirred at 65° C. for 72 hr to obtain compound 31.

### Example 34

### Preparation of N-(9-cyclopentyl-8-(4-methoxyphenyl)-9H-purin-6-yl)-4-methylbenzenesulfonamide (32)

The following compound was made by general procedure D using 31 (0.10 g, 0.32 mmol), dry DMF (5 mL), NaH (15.36 mg, 0.64 mmol), 4-methylbenzene-1-sulfonyl chloride (0.091 gm, 0.48 mmol) to obtain compound 32. 1H NMR (400 MHz, Chloroform-d) δ 8.20 (s, 1H), 7.98 (s, 2H), 7.54 (d, J=8.8 Hz, 2H), 7.24 (d, J=8.0 Hz, 3H), 7.01 (d, J=8.8 Hz, 2H), 4.74 (p, J=8.8 Hz, 1H), 3.86 (s, 3H), 2.49-2.39 (m, 2H), 2.37 (s, 3H), 2.11-1.96 (m, 4H), 1.67-1.57 (m, 2H).

### Example 35

### Preparation of 9-butyl-8-(4-methoxyphenyl)-9H-purin-6-amine (33)

To a suspension of 28 (0.5 gm, 2.07 mmol) in 8 mL of DMF was added K₂CO₃ (0.572 gm, 4.14 mmol). The reaction mixture was stirred at 60° C. for 30 min. A solution of bromobutane (0.33 mL, 3.10 mmol) in 3 mL DMF was added to the reaction mixture and the mixture was stirred at 65° C. for 72 hr to obtain compound 33.

### Example 36

### Preparation of N-(9-butyl-8-(4-methoxyphenyl)-9H-purin-6-yl)-4-methylbenzenesulfonamide (34)

The following compound was made by general procedure D using 33 (0.10 g, 0.33 mmol), dry DMF (5 mL), NaH (15.84 mg, 0.66 mmol), 4-methylbenzene-1-sulfonyl chloride (0.094 gm, 0.49 mmol) to obtain compound 34.

### Example 37

### Preparation of 8-(4-methoxyphenyl)-9-(1-methylpiperidin-4-yl)-9H-purin-6-amine (35)

To a suspension of 28 (0.5 gm, 2.07 mmol) in 8 mL of DMF was added K₂CO₃ (0.572 gm, 4.14 mmol). The reaction mixture was stirred at 60° C. for 30 min. A solution of 4-Bromo-1-methylpiperidine (0.55 mg, 3.10 mmol) in 3 mL DMF was added to the reaction mixture and the mixture was stirred at 65° C. for 72 hr to obtain compound 35.

### Example 38

### Preparation of N-(8-(4-methoxyphenyl)-9-(1-methylpiperidin-4-yl)-9H-purin-6-yl)-4-methylbenzenesulfonamide (36)

The following compound was made by general procedure D using 35 (0.10 g, 0.29 mmol), dry DMF (5 mL), NaH (13.92 mg, 0.58 mmol), 4-methylbenzene-1-sulfonyl chloride (0.083 gm, 0.43 mmol) to obtain compound 36.

### Example 39

### Preparation of N-(8-(4-methoxyphenyl)-9H-purin-6-yl)-4-methylbenzenesulfonamide (37)

The following compound was made by general procedure D using 28 (0.10 g, 0.41 mmol), dry DMF (5 mL), NaH (19.68 mg, 0.82 mmol), 4-methylbenzene-1-sulfonyl chloride (0.118 gm, 0.62 mmol) to obtain compound 37.

### Example 40

### Preparation of 8-(4-fluorophenyl)-9-methyl-9H-purin-6-amine (38)

Compound 3 (0.5 gm, 1.95 mmole), 4-fluorophenylboronicacid (0.465 gm, 2.92 mmole), K2CO3 (0.541 gm, 3.9 mmole) were taken in a pressure tube and dissolved in (9:1) mixture of Dioxane & water (10 mL). Solution was purged with Argon gas for 30 minutes. Then catalyst Pd(PPh3)4 (0.226 gm, 0.19 mmole) was added and the reaction mixture was stirred at 100° C.-110° C. for overnight. Reaction was monitored by checking TLC. After completion, reaction mass was washed with water and extracted with ethyl acetate and evaporated. The product was then purified by column chromatography (Silica gel, mesh size 100-200) eluting (5% MeOH/CHCl3) to obtain 38 as pale yellow amorphous solid (0.436 g, 77%).

### Example 41

### Preparation of N-(8-(4-fluorophenyl)-9-methyl-9H-purin-6-yl)-4-methylbenzenesulfonamide (39)

The following compound was made by general procedure D using 38 (0.10 g, 0.35 mmol), dry DMF (5 mL), NaH (18.81 mg, 0.70 mmol), 4-methylbenzene-1-sulfonyl chloride (0.085 gm, 0.42 mmol) to obtain compound 39 (0.052 g, 32%) as off white solid. 1H NMR (400 MHz, DMSO-d6) δ 8.33 (s, 1H), 7.90-7.87 (m, 2H), 7.85-7.78 (m, 2H), 7.42-7.36 (m, 2H), 7.35-7.28 (m, 2H), 3.78 (s, 3H), 2.31 (s, 3H).

### Example 42

Preparation of 8-(4-chlorophenyl)-9-methyl-9H-purin-6-amine (40)

Compound 3 (0.5 gm, 1.95 mmole), 4-chlorophenylboronicacid (0.490 gm, 2.92 mmole), K2CO3 (0.541 gm, 3.9 mmole) were taken in a pressure tube and dissolved in (9:1) mixture of Dioxane & water (10 mL). Solution was purged with Argon gas for 30 minutes. Then catalyst Pd(PPh3)4 (0.226 gm, 0.19 mmole) was added and the reaction mixture was stirred at 100° C.-110° C. for overnight. Reaction was monitored by checking TLC. After completion, reaction mass was washed with water and extracted with ethyl acetate and evaporated. The product was then purified by column chromatography (Silica gel, mesh size 100-200) eluting (5% MeOH/CHCl3) to obtain 40 as pale yellow amorphous solid (0.452 g, 75%).

### Example 43

### Preparation of N-(8-(4-chlorophenyl)-9-methyl-9H-purin-6-yl)-4-methylbenzenesulfonamide (41)

The following compound was made by general procedure D using 40 (0.10 g, 0.37 mmol), dry DMF (5 mL), NaH (19.63 mg, 0.74 mmol), 4-methylbenzene-1-sulfonyl chloride (0.088 gm, 0.44 mmol) to obtain compound 41 (0.046 g, 36%) as off white solid. 1H NMR (400 MHz, DMSO-d6) δ 8.34 (s, 1H), 7.88-7.76 (m, 4H), 7.60 (d, J=8.4 Hz, 2H), 7.31 (d, J=8.0 Hz, 2H), 3.79 (s, 3H), 2.31 (s, 3H).

### Example 44

### Preparation of 4-(6-amino-9-methyl-9H-purin-8-yl)benzonitrile (42)

Compound 3 (0.5 gm, 1.95 mmole), 4-cyanophenylboronicacid (0.510 gm, 2.92 mmole), K2CO3 (0.541 gm, 3.9 mmole) were taken in a pressure tube and dissolved in (9:1) mixture of Dioxane & water (10 mL). Solution was purged with Argon gas for 30 minutes. Then catalyst Pd(PPh3)4 (0.226 gm, 0.19 mmole) was added and the reaction mixture was stirred at 100° C.-110° C. for overnight. Reaction was monitored by checking TLC. After completion, reaction mass was washed with water and extracted with ethyl acetate and evaporated. The product was then purified by column chromatography (Silica gel, mesh size 100-200) eluting (3% MeOH/CHCl3) to obtain 42 as pale yellow amorphous solid (0.410 g, 72%).

### Example 45

### Preparation of N-(8-(4-cyanophenyl)-9-methyl-9H-purin-6-yl)-4-methylbenzenesulfonamide (43)

The following compound was made by general procedure D using 42 (0.10 g, 0.39 mmol), dry DMF (5 mL), NaH (17.43 mg, 0.78 mmol), 4-methylbenzene-1-sulfonyl chloride (0.078 gm, 0.46 mmol) to obtain compound 43 (0.046 g, 36%) as off white solid. 1H NMR (400 MHz, Chloroform-d) δ 8.25 (s, 1H), 7.89-7.84 (m, 2H), 7.83-7.80 (m, 2H), 7.77-7.72 (m, 2H), 7.19-7.15 (m, 2H), 3.81 (s, 3H), 2.28 (s, 3H)

### Biological Assay

T cells are part of adaptive immune system and serve as cellular effectors during infection and cancer. Upon priming and activation, coordinated metabolic rewiring and epigenetic remodeling seems to have utmost importance to render productive T cell response. The mammalian target of rapamycin (mTOR), an evolutionary conserved serine/threonine kinase, has been reported integrating immune signals and metabolic cues for proper maintenance and activation of T cells. Furthermore, activation and regulation of mTOR signaling pathway orchestrates T cell metabolism, with subsequent impact on T cell effector responses. mTOR exists in two multiprotein complexes which mainly differ in their adaptive protein counterpart. mTOR complex 1 (mTORC1) has a scaffolding protein named as regulatory associated protein of mTOR (RAPTOR) which is a target for the immunosuppressant rapamycin. But contrastingly, rapamycin insensitive companion of mTOR (RICTOR), a scaffolding protein of mTORC2, is relatively resistant to rapamycin. By date, mTORC1 is best known for promoting translation initiation and protein synthesis by directly phosphorylating ribosomal protein S6 kinases (S6Ks) and eIF4E-binding protein (4E-BP). Here, we have identified a potent sulfonamide derivative which potentiates CD8⁺ T cell effector function by improving their metabolic fitness in a mTORC1 dependent manner.

### Materials & Methods

### Example 44

### Animal Studies

Splenic CD3⁺ T cells were obtained from C57BL/6 mice with an age of 6 to 8 weeks. Equal numbers of male and female mice were randomly assigned for each experiment to omit the influence of sexual biasness on the experimental results. All animal experiments were approved by the ethical review body of CSIR-Indian Institute of Chemical biology. Animals were maintained under specific pathogen-free conditions of in-house animal facility.

### Example 45

### Human Studies

Human peripheral blood mononuclear cells (PBMC) were obtained from fully anonymized whole blood bags (age & sex related information were not provided) collected from healthy volunteer blood donors at NRS Medical College & Hospital, Kolkata, India. All volunteers signed a consent form, and all studies were approved by the CSIR-Indian Institute of Chemical biology Ethics Committee.

### Example 46

### CD3⁺ T Cell Isolation and Culture

CD3⁺ T cells were isolated from human peripheral blood by density-gradient (Ficoll gradient) centrifugation followed by a negative selection using Dynabeads Untouched Human T Cells Kit, resulting in a typically >90% pure CD3⁺ T cell population.

Murine CD3⁺ T cells were isolated from wild-type C57BL/6 spleens by negative selection using Dynabeads Untouched Mouse T Cells Kit which also resulted in a typically >90% pure murine CD3⁺ T cell population.

Purified naïve CD3⁺ T cells were activated either in presence of Comp 19 (10M ml⁻¹) or vehicle (DMSO) with plate-bound anti-CD3 (5 g ml⁻¹) and anti-CD28 (2 g ml⁻¹) for 72 h in RPMI-1640 medium containing 10% FBS, 50 M R-ME, 50 U/mL penicillin and 50 mg/mL streptomycin and 100 IUmL⁻¹ rIL-2.

### Example 47

### Flow Cytometry Analysis of Protein Expression, Cytokine Production and Proliferation

Cell surface protein expression was assessed by flow cytometry (FACS) staining with specific monoclonal antibodies at 1:200 dilutions in FACS buffer (0.5% BSA, 0.1% Sodium azide in PBS) for 20 minutes at 4° C. prior to washing and analysis.

### Example 48

Measurement of cytokine production: To assess IFN-γ and TNF-α secretion by intracellular cytokine staining, cells (0.2×10⁶) were treated for 4 hours with restimulation cocktail containing phorbolmyristate acetate (50 ng ml⁻¹) and ionomycin (500 ng ml⁻¹), brefeldin A (to block anterograde protein transport). Then cells were stained for surface markers and simultaneously labeled with Live/Dead Yellow Fluorescent Dye for 20 min at 4° C. Next cells were washed with PBS, fixed for 20 min at 4° C. with fixation/permeabilization solution, washed with permeabilization buffer, finally stained for 30 min with anti-IFN-γ and anti-TNF-α mAb in dark at 4° C., washed with PBS and analyzed.

### Example 49

pS6 Staining: Intracellular phospho ribosomal protein S6 (pS6) were stained using Transcription factor Phospho Buffer set. Single cell suspension was prepared and cells were washed with 1×PBS. Next cells were fixed and permeabilized with BD Pharmingen™ Transcription Factor Phospho (TFP) Buffer Set for 50 mins at 4° C. After fixation, cells were washed using 1×TFP Perm/Wash Buffer prior to incubation with BD Phosflow™ Perm Buffer III for 30 mins at 4° C. Next cells were washed two times with 1×TFP Perm/Wash Buffer and later stained with fluorescent antibodies for 20 min at 4° C., dark condition. Finally, cells were washed, dissolved in PBS and analysed by flow cytometry.

### Example 50

Glucose uptake assay: Glucose uptake by activated T cells was evaluated using fluorescent labeled deoxy-glucose analogue, 2NBDG. Activated T cells (0.2×10⁶) were washed two times, resuspended in glucose-free media containing 2NBDG (20 M ml⁻¹), incubated for 30 min at 37° C. Later cells were washed, stained for surface protein using specific monoclonal antibody against CD8, acquired in BD LSR Fortessa machine.

### Example 51

### Western Blotting Analysis

Cell lysates from 4×10⁶ cells per condition were prepared in RIPA buffer containing protease inhibitor cocktail. Protein concentrations were determined with a serial dilution standard curve of Bradford assay. 10% SDS-PAGE was prepared to resolve the whole cell lysate and was transferred onto PVDF membrane. The membranes were then incubated with respective primary antibodies overnight at 4° C. Next day membranes were washed with TBST, incubated with HRP conjugated anti rabbit IgG at room temperature for 2 hrs. Membranes were then washed with TBST and ECL clarity max solution (Bio-Rad Clarity Western ECL Substrate) was used for band detection.

### Example 52

### Adoptive Transfer Model:

EL-4.OVA tumor cells (1×10⁶) or B16-F10 melanoma (0.5×10⁶ cells) were injected subcutaneously into the left flank of 7-10 weeks old C57BL/6 mice. After 9 days of tumor establishment, ex vivo activated OT-1 T cells at 0.5×10⁶ cells/mouse were adoptively transferred intravenously into tumor-bearing recipients.

### Lipophilicity Assay

### Assay Procedure

1.56 g NaH₂PO₄O·2H₂O was dissolved in 0.5 L water in a 1 L beaker. After adjusting pH to 7.4 using NaOH solution, the volume was made up to 1 L. Equal volumes of sodium phosphate buffer (10 mM, pH 7.4) and n-octanol were added to a separation funnel and mixed thoroughly by shaking and inverting the funnel several times. The two layers were allowed to separate for overnight and then dispensed in two separate glass bottles. 10 mM stock solution was prepared in 100% DMSO and stored at 4° C. 495 μL of organic phase (1-octanol) was added to each well of a 2 mL deep well plate, followed by 495 μL of buffer and 10 μL of test substance was added. The plate was incubated for 3 hr at room temperature on a plate shaker at 500 rpm. After incubation, the samples were allowed to equilibrate for 20 min and then centrifuged at 4000 rpm for 30 min for complete phase separation and analysed by LC-UV.

Log D=Log (area of octanol/area of buffer)

TABLE 5

| S1 | | | LogD @ |
|---|---|---|---|
| No | Compound No | Structure | pH 7.4 |
| 1 | 19 | 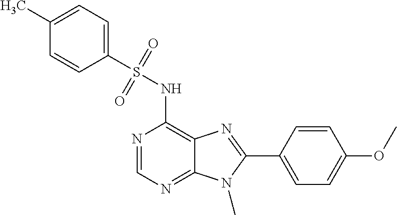 | 0.96 |
| 2 | 22 | 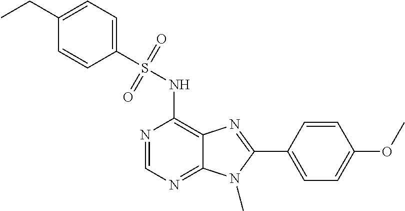 [SMILES: CCc1ccc(S(=O)(=O)Nc2ncnc3c2nc(-c2ccc(OC)cc2)n3C)cc1] | 4.32 |
| 3 | Ketoconazole(Control) | NA | 3.10 |
| 4 | Metroprolol(Control) | NA | −0.34 |
| 5 | Propranolol | NA | 1.04 |
| | (Control) | | |

Log D Criteria: <0: Low lipophilicity; 0-1: Moderate lipophilicity; >2: High lipophilicity

### Plasma Stability Assay:

### Assay Procedure

1 mM Stock of test compound was prepared from 10 mM initial stock solution of compounds by diluting 10 μL of 10 mM stock with 90 μL of DMSO. Then 10 μL of 1 mM stock was diluted with 90 μL of DMSO to give 100 μM concentration. The frozen plasma was thawed at room temperature and centrifuged at 1400 rpm at 4° C., for 15 minutes. Approximately 90% of the clear supernatant fraction was transferred to a separate tube and was used for the assay. Final working stock of 1 μM was prepared by diluting 3 μL of 100 μM with 297 μL of plasma. Plasma containing the test compound was incubated for 120 mins at 37° C. in shaker with 500 rpm. 50 μL of aliquot of sample at 0, 15, 30, 60 and 120 minutes were precipitated with 150 μL of acetonitrile containing internal standard and centrifuged at 4000 rpm at 4° C. for 20 minutes. 120 μL of supernatant was diluted with 120 μL of water and analyzed by LC-MS/MS.

TABLE 6

| | | | Mean % remaining |
|---|---|---|---|
| S1 | | | at 2 hrs in human |
| No | Compound No | Structure | Plasma |
| 1 | 19 | 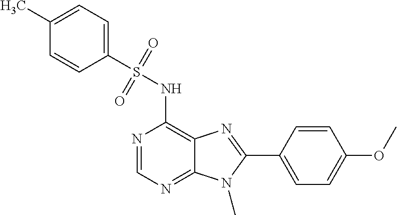 | 85.81 |
| 2 | 22 | 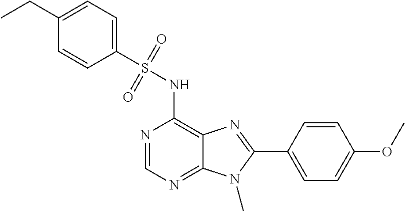 [SMILES: CCc1ccc(S(=O)(=O)Nc2ncnc3c2nc(-c2ccc(OC)cc2)n3C)cc1] | 3.90 |
| 3 | Propantheline(Control) | NA | 0.33 |

### In-Vitro Evaluation of Metabolic Stability Using Human Liver Microsomes (Human Liver Microsomal Stability-HLM)

### Assay Procedure

1 mM stock solution of test compound was prepared in DMSO and diluted with Acetonitrile:Water (1:1) to get a 100 μM working concentration. 100 mL of Milli Q water was added to K₂HPO₄ (1.398 g) and KH₂PO₄ (0.27 g) to get final pH 7.4 solution of potassium phosphate buffer. 3.333 mg/mL microsomal suspension was prepared by diluting 499.95 μL of 20 mg/mL microsomal stock to 2500.05 μL with buffer. 532.5 μL of 16 mM NADPH stock was added to 2467.5 μL of potassium phosphate buffer to get 2.84 mM working stock. 75 μL of 3.333 mg/mL working stock of liver microsomes and 85 μL of buffer was added to 2.5 μL of test compounds (100 μM). The above mixture was pre incubated for 15 minutes at 37° C. After pre incubation, 32.5 μL of the mixture was added to 17.5 μL of buffer, this was incubated for 60 minutes at 37° C. [60 min Without Cofactor (NADPH)]. 16.25 μL of the pre incubated mixture and 8.75 μL of cofactor was added to 150 μL of acetonitrile containing internal standard [0 min Sample]. 62 μL of cofactor was added to remaining pre incubation mixture [Incubation mixture]. 25 μL of incubation mixture at 0, 5, 15, 30, 60 min and 60 min without cofactor were precipitated with 150 μL of acetonitrile containing internal standard, vortexed and centrifuged at 4000 rpm at 4° C. for 20 minutes. 120 μL of supernatant was diluted with 120 μL of water and analyzed by LC-MS/MS [sample preparation].

TABLE 7

| S1 | | | % QH (Well |
|---|---|---|---|
| No | Compound No | Structure | stirred model) |
| 1 | 19 | 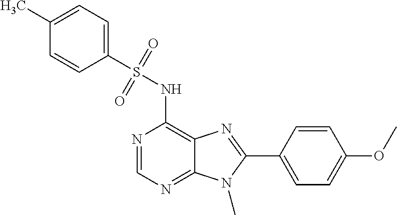 | 0.77 |
| 2 | 22 | 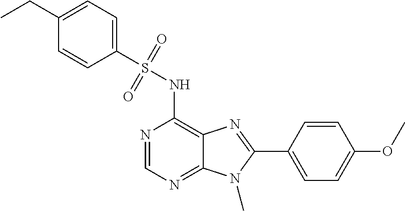 [SMILES: CCc1ccc(S(=O)(=O)Nc2ncnc3c2nc(-c2ccc(OC)cc2)n3C)cc1] | 31.77 |
| 3 | Verapamil HCl (Control) | NA | 89.23 |

Classifications criteria: % QH; <30: Low Clearance 30-70: Medium Clearance >70: High Clearance

### Advantages of the Invention

The various advantage of the present invention is:
a) The 8-phenyl-purine-6-amino compounds with structure I is capable to increase the effector function (IFNγ production) of T cells and hence heightened their anti-tumor response.
b) The 8-phenyl-purine-6-amino compounds with structure I can be used to activate of mTOR.
c) The 8-phenyl-purine-6-amino compounds with structure I used in modulating the expression of immune checkpoint inhibitors (PD1, Tim3, Lag3, CD38) on T cells.
d) The present invention provides a method to establish relation between mTOR activation and optimal T-cell effector function.
e) The present invention provides a method to evaluate the efficacy of mTOR activator in generating T cells with improved anti-tumor response in preclinical models.
f) The present invention provides a method to evaluate the efficacy of mTOR activator-induced anti-tumor T cells in generating long-lasting anti-tumor response in tumor bearing mice.

1-11. (canceled)

12. An 8-phenyl-purine-6-amino compound having structure I or salts thereof, 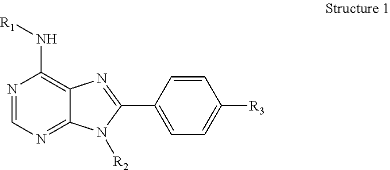
wherein:
R₁ is independently selected from the group consisting of: 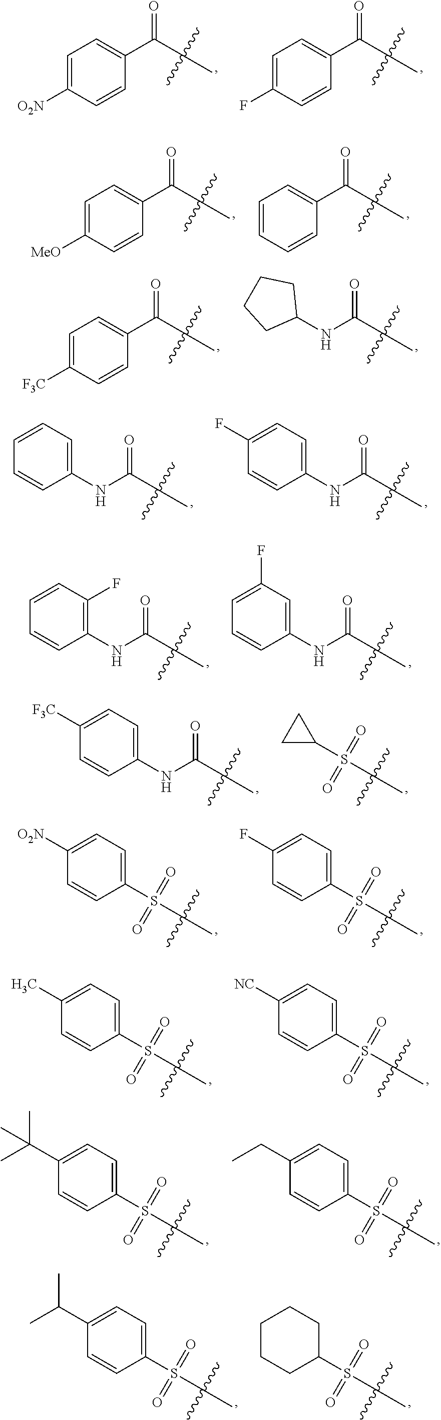 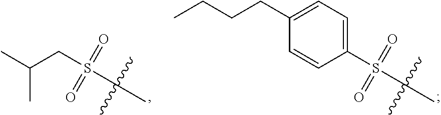
R₂ is independently selected from the group consisting of: 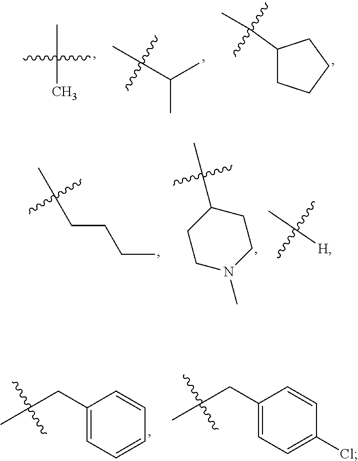
and
R₃ is independently selected from the group consisting of:
—OMe, —Br, —Cl, —CF₃, —OCF₃, —CH₃, —F, —CN.

13. The compound according to claim 12, wherein the compound is selected from the group consisting of:
N-(8-(4-methoxyphenyl)-9-methyl-9H-purin-6-yl)-4-nitrobenzamide (5),
4-fluoro-N-(8-(4-methoxyphenyl)-9-methyl-9H-purin-6-yl)benzamide (6),
4-methoxy-N-(8-(4-methoxyphenyl)-9-methyl-9H-purin-6-yl)benzamide (7),
N-(8-(4-methoxyphenyl)-9-methyl-9H-purin-6-yl)-4-(trifluoromethyl)benzamide (9),
1-(8-(4-methoxyphenyl)-9-methyl-9H-purin-6-yl)-3-phenylurea (11),
1-(4-fluorophenyl)-3-(8-(4-methoxyphenyl)-9-methyl-9H-purin-6-yl)urea (12),
1-(2-fluorophenyl)-3-(8-(4-methoxyphenyl)-9-methyl-9H-purin-6-yl)urea (13),
1-(3-fluorophenyl)-3-(8-(4-methoxyphenyl)-9-methyl-9H-purin-6-yl)urea (14),
1-(8-(4-methoxyphenyl)-9-methyl-9H-purin-6-yl)-3-(4-(trifluoromethyl)phenyl)urea (15),
N-(8-(4-methoxyphenyl)-9-methyl-9H-purin-6-yl)-4-nitrobenzenesulfonamide (17),
4-fluoro-N-(8-(4-methoxyphenyl)-9-methyl-9H-purin-6-yl)benzenesulfonamide (18),
N-(8-(4-methoxyphenyl)-9-methyl-9H-purin-6-yl)-4-methylbenzenesulfonamide (19),
4-cyano-N-(8-(4-methoxyphenyl)-9-methyl-9H-purin-6-yl)benzenesulfonamide (20),
4-(tert-butyl)-N-(8-(4-methoxyphenyl)-9-methyl-9H-purin-6-yl)benzenesulfonamide (21),
4-ethyl-N-(8-(4-methoxyphenyl)-9-methyl-9H-purin-6-yl)benzenesulfonamide (22),
4-isopropyl-N-(8-(4-methoxyphenyl)-9-methyl-9H-purin-6-yl)benzenesulfonamide (23),
4-butyl-N-(8-(4-methoxyphenyl)-9-methyl-9H-purin-6-yl)benzenesulfonamide (26),
N-(9-isopropyl-8-(4-methoxyphenyl)-9H-purin-6-yl)-4-methylbenzenesulfonamide (30),
N-(9-cyclopentyl-8-(4-methoxyphenyl)-9H-purin-6-yl)-4-methylbenzenesulfonamide (32),
N-(9-butyl-8-(4-methoxyphenyl)-9H-purin-6-yl)-4-methylbenzenesulfonamide (34),
N-(8-(4-methoxyphenyl)-9-(1-methylpiperidin-4-yl)-9H-purin-6-yl)-4-methylbenzenesulfonamide (36),
N-(8-(4-methoxyphenyl)-9H-purin-6-yl)-4-methylbenzenesulfonamide (37),
N-(8-(4-fluorophenyl)-9-methyl-9H-purin-6-yl)-4-methylbenzenesulfonamide (39),
N-(8-(4-chlorophenyl)-9-methyl-9H-purin-6-yl)-4-methylbenzenesulfonamide (41),
N-(8-(4-cyanophenyl)-9-methyl-9H-purin-6-yl)-4-methylbenzenesulfonamide (43),
N-(9-benzyl-8-(4-methoxyphenyl)-9H-purin-6-yl)-4-methylbenzenesulfonamide (44) and
N-(9-(4-chlorobenzyl)-8-(4-methoxyphenyl)-9H-purin-6-yl)-4-methylbenzenesulfonamide (45).

14. A process for preparation the compound according to claim 13, comprising:
(i) preparing a reaction mixture by adding Adenine (compound 1) 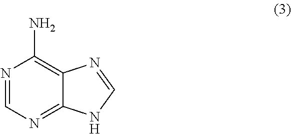
in dioxane and water mixture (1:1);
(ii) adding NaOH and TBAF into the reaction mixture of step (i);
(iii) adding dropwise Mel to the reaction mixture after step (ii) at 0° C. temperature and allowing the reaction mixture to stir at room temperature for overnight to obtain a compound 2 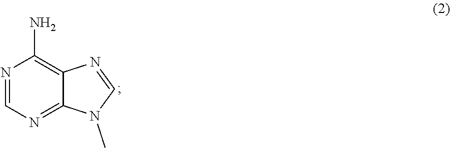
(iv) dissolving the compound 2 obtained in step (iii) in a mixture of dioxane and 0.5(M) NaOAc solution (9:1);
(v) adding dropwise liquid bromine into the reaction mixture of step (iv) and stirring at room temperature for overnight to obtain a compound 3 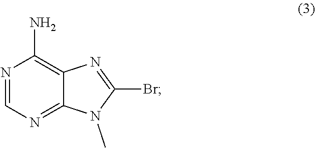
(vi) preparing a reaction mixture of the compound 3 obtained in step (v) with 4-methoxy phenyl boronic acid and K₂CO₃ in a pressure tube followed by dissolving the reaction mixture in dioxane and water (9:1) and purging the mixture with argon gas for 30 minutes;
(vii) adding a catalyst Pd(PPh₃)₄ into the reaction mixture obtained in step (vi) and stirring the mixture at a temperature of 100° C.-110° C. for overnight to obtain compounds 4, 38, 40, 42 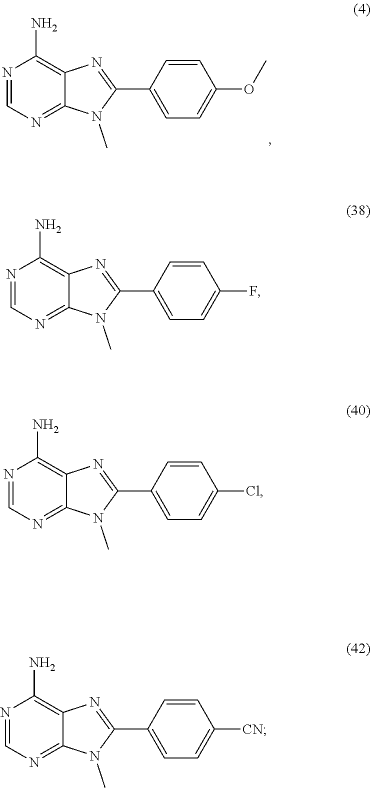 [SMILES: COc1ccc(-c2nc3c(N)ncnc3n2C)cc1 ; Cn1c(-c2ccc(F)cc2)nc2c(N)ncnc21 ; Cn1c(-c2ccc(Cl)cc2)nc2c(N)ncnc21 ; Cn1c(-c2ccc(C#N)cc2)nc2c(N)ncnc21]
(viii) reacting the compound 4 obtained in step (vii) with LiHMDS as a base followed by reaction with an acid chloride in dry THF at 0° C. temperature for 30 minutes to obtain the compound selected from the group consisting of 5, 6, 7, 8, 9;
(ix) reacting the compound 4 obtained in step (vii) with LiHMDS as a base followed by reaction with an isocyanate in dry THF at 0° C. temperature for 30 minutes to obtain the compound selected from the group consisting of 10, 11, 12, 13, 14, 15;
(x) reacting the compound 4 obtained in step (vii) with NaH as a base followed by reaction with an sulfonyl chloride in dry DMF at 0° C. temperature for 30 minutes to obtain the compound selected from the group consisting of 16, 17, 18, 19, 20, 21, 22, 23, 24, 25, 26;
(xi) reacting the compounds 38, 40, 42 obtained in step (vii) with NaH as a base followed by reaction with 4-methylbenzene-1-sulfonyl chloride in dry DMF at 0° C. temperature for 30 minutes to obtain the compound selected from the group consisting of 39, 41, 43;
(xii) preparing the reaction mixture by adding adenine (Compound 1) in dry DMF followed by adding K₂CO₃;
(xiii) adding aliphatic or aromatic halide into the reaction mixture of step (xii) and stirring the mixture for 12 hrs to obtain compounds 1A, 1C, 1E 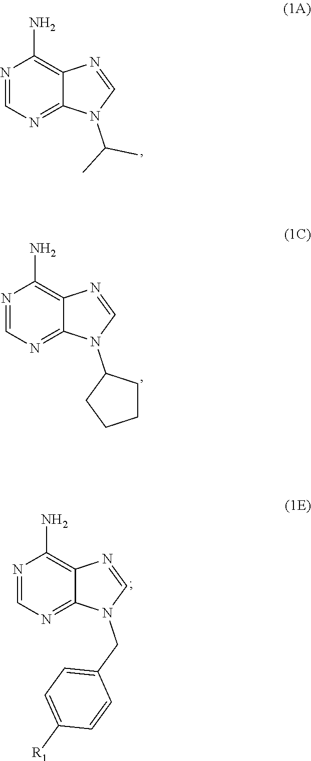
(xiv) reacting the compounds 1A, 1C, 1E obtained in step (xiii) with NBS in dry CHCl₃ for 12 hrs at room temperature to obtain a compound 1B, 1D, 1F 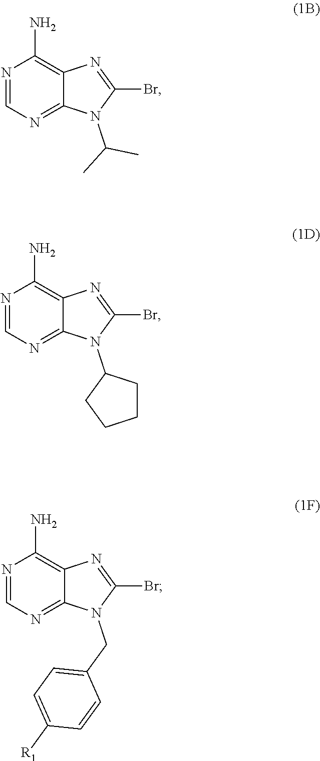
(xv) preparing a reaction mixture of the compounds 1B, 1D, 1F obtained in step (xiv), 4-methoxy phenyl boronic acid and K₂CO₃ in a pressure tube followed by dissolving the reaction mixture in (9.1) mixture of dioxane and water and purging mixture with argon gas for 30 minutes;
(xvi) adding the catalyst Pd(PPh₃)₄ into the reaction mixture obtained in step (xv) and stirring the mixture at 100° C.-110° C. for overnight to obtain compounds 29, 31, 1G 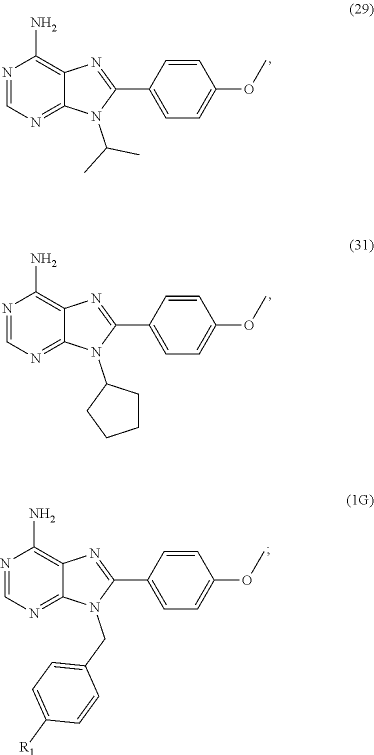
and
(xvii) reacting the compound 29, 31, 1G obtained in step (xvi) with NaH as a base followed by reaction with 4-methylbenzene-1-sulfonyl chloride in dry DMF at 0° C. temperature for 30 minutes to obtain the compound selected from the group consisting of 30, 32, 44, 45.

15. The process according to claim 14, wherein the acid chloride of step (viii) is selected from the group consisting of 4-nitrobenzoyl chloride, 4-fluorobenzoyl chloride, 4-methoxybenzoyl chloride, benzoyl chloride and 4-(trifluoromethyl)benzoyl chloride.

16. The process according to claim 14, wherein the isocyanate of step (ix) is selected from the group consisting of isocyanatocyclopentane, Phenyl isocyanate, 4-Fluorophenyl isocyanate, 2-Fluorophenyl isocyanate, 3-Fluorophenyl isocyanate, and 4-(Trifluoromethyl)phenylisocyanate.

17. The process according to claim 14, wherein the sulfonyl chloride of step (x) is selected from the group consisting of cyclopropanesulfonyl chloride, 4-nitrobenzene-1-sulfonyl chloride, 4-fluorobenzene-1-sulfonyl chloride, 4-methylbenzene-1-sulfonyl chloride, 4-cyanobenzene-1-sulfonyl chloride, 4-(tert-butyl)benzene-1-sulfonyl chloride, 4-ethylbenzene-1-sulfonyl chloride, 4-isopropylbenzene-1-sulfonyl chloride, cyclohexanesulfonyl chloride, 2-methylpropane-1-sulfonyl chloride, and 4-butylbenzene-1-sulfonyl chloride.

18. The process according to claim 14, wherein the aliphatic or aromatic halide of step (xiii) is selected from the group consisting of isopropyl iodide, cyclopentyl bromide and benzyl bromide, 4-chlorobenzyl bromide.

19. The compound according to claim 12, wherein the compound enhances effector function of T cells and anti-tumor response.

20. The compound according to claim 12, wherein the compound modulates the expression of one or more immune checkpoint inhibitors selected from PD1, Tim3, Lag3, and CD38 on T cells.

21. The compound according to claim 12, wherein the compound provides a method to evaluate and establish relation between mTOR activation and optimal T-cell effector function.

22-23. (canceled)

24. The compound according to claim 12, wherein the compound induces anti-tumor T cells and generates long-lasting anti-tumor response in tumor bearing mice.

25. The compound according to claim 12, wherein the compound enhances T cell effector functionality and in T cell activation in cancer immunotherapy, wherein cancer immunotherapy comprises adoptive T cell therapy (ACT).
